(19) United States
(12) Patent Application Publication
(10) Pub. No.: US 2010/0272783 A1
(43) Pub. Date: Oct. 28, 2010

(54) Methods of Treating Infections of the Nail

(75) Inventors: Lu Wang, Emeryville, CA (US); Bahram Memarzadeh, San Carlos, CA (US)

Correspondence Address: Hamilton DeSanctis & Cha, LLP, 3239 El Camino Real, Suite 220, Palo Alto, CA, 94306, US

(73) Assignee: NovaBay Pharmaceuticals, Inc.

(21) Appl. No.: 12/766,673

(22) Filed: Apr. 23, 2010

Related U.S. Application Data
(60) Provisional application No. 61/172,688, filed on Apr. 24, 2009.

(51) Int. Cl. A61K 9/70; A61K 31/185; A61P 17/00; A61P 43/00

(52) U.S. Cl. 424/447; 514/578

(57) ABSTRACT

Disclosed herein are compositions and methods of treating or preventing an infection of the nail, claw, or hoof comprising administering to a subject a pharmaceutical composition comprising an N-halogenated or N,N-dihalogenated amine compound of the application.

### RELATED APPLICATION

This application claims the benefit of U.S. Provisional Patent Application No. 61/172,688, filed on Apr. 24, 2009, entitled “Methods of Treating Infections of the Nail” which is incorporated herein by reference.

### FIELD

This invention relates to methods of treating or preventing infections of the nail, claw, or hoof, comprising administering an active agent comprising an N-halogenated or N,N-dihalogenated amine compound to a subject in need thereof.

### BACKGROUND

Fungal infection of the nail, also referred to by the terms “onychomycosis” and “tinea unguium” affect approximately 5% of the population worldwide, including 2-13% of the population of North America and Europe. Infection rates may be higher in subjects with HIV infection, diabetes, or who have suppressed immunological responses to fungi. Onychomycosis is caused most commonly by dermatophytes, and less commonly by molds and yeasts, such as yeasts of the genus Candida. Onychomycosis can be categorized into several varieties, including distal and lateral subungual onychomycosis, endonyx onychomycosis, white superficial onychomycosis, proximal subungual onychomycosis, Candida onychomycosis, and total dystrophic onychomycosis. Nails may also be infected by certain bacteria or virus. Similar infections of the claw may affect animals such as cats, dogs, birds, and reptiles, and similar infections of the hoof may affect animals such as horses, cattle, goats, pigs, and sheep.

Onychomycosis is presently treated primarily with oral antifungal agents. Oral antifungal agents include terbinafine (e.g. Lamisil®), itraconazole (e.g. Sporanox®), and fluconazole (e.g. Diflucan®). Such compounds can cause hepatic injury, however, and monitoring of liver enzymes may be required during treatment. Most topical agents such as Penlac® and Loceryl® tend to be less effective than the oral agents, except in mild cases that mainly affect the distal portion of the nail plate.

Existing treatments of nail infections such as onychomycosis have either high risks of adverse effects or limited efficacy, in addition to other drawbacks such as drug interactions. Thus, there is a need for new treatments for onychomycosis and other infections of the nail, hoof, that are effective, safe, and convenient to use.

### SUMMARY

This disclosure describes methods of treating or preventing infections of the nail, claw, or hoof, comprising administering an active agent comprising an N-halogenated or N,N-dihalogenated amine compound to a subject in need thereof.

In one aspect, the N-halogenated or N,N-dihalogenated amine compound may be a compound of Formula (I)

A-C(R¹R²)R(CH₂)ₙC(R³R⁴)—Y—Z (I)

or a derivative thereof, wherein

A is hydrogen, HalNH— or Hal₂N—, wherein Hal is a halogen selected from the group consisting of chloro, bromo and iodo;

R¹ is hydrogen or an optionally substituted group selected from the group consisting of alkyl, cycloalkyl, heteroalkyl, aryl, heteroaryl, and heterocycloalkyl groups, and —COOH;

R² is hydrogen or an optionally substituted group selected from the group consisting of alkyl, cycloalkyl, heteroalkyl, aryl, heteroaryl, and heterocycloalkyl groups, or R¹ and R² together with the carbon atom to which they attach form an optionally substituted cycloalkyl or heterocycloalkyl group;

R is a carbon-carbon single bond or a divalent cycloalkylene radical with three to six carbon atoms,

n is 0 or an integer from 1 to 13;

R³ and R⁴ are each independently selected from the group consisting of hydrogen, fluoro, —NHHal, NHal₂, and an optionally substituted group selected from the group consisting of alkyl, cycloalkyl, heteroalkyl, aryl, heteroaryl, and heterocycloalkyl groups;

Y is selected from a group consisting of a single bond; —O—, —CF₂—, —CHF—, —C(═O)—, —C(═O)O—, —OC(═O)—, —C(═O)NR^{a}—, —NR^{a}C(═O)—, —P(═O)(OR^{b})O—, —OP(═O)(OR^{b})—, —P(═O)(OR^{b})NR^{c}—, —NR^{c}P(═O)(OR^{b})—, —S(═O)₂, —S(═O)₂O—, —OS(═O)₂—, —S(═O)₂NR^{d}—, —NR^{d}S(═O)₂—, or heteroaryl wherein R^{a}, R^{b}, R^{c} and R^{d} are each independently selected from the group consisting of hydrogen, and optionally substituted alkyl, aryl, cycloalkyl, heteroalkyl, heteroaryl, and heterocycloalkyl; a divalent (C₁₋₁₈)alkyl group in which, optionally, one or two methylene groups are replaced with a mono- or di-substituted methylene group; and a divalent (C₁₋₁₈)heteroalkyl group wherein the divalent (C₁₋₁₈)heteroalkyl group is a divalent (C₁₋₁₈)alkyl group in which, optionally, one or two methylene groups are replaced with 1 or 2-NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂—, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, and (C₆₋₁₀)aryl(C₁₋₄)alkylC(═O)—wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄) alkyl, the heterocycloalkyl group containing 2-10 carbon atoms and 1 to 4 heteroatoms selected from N, O or S;

Z is selected from the group consisting of hydrogen, —CO₂H, —CONH₂, —SO₃H, —SO₂NH₂, —P(═O)(OH)₂, B(OH)₂, —[X(R⁵)(R⁶)R⁷]Q, —S(═O)₂NR^{c}R^{d}, —S(═O)₂NHC(═O)R^{e}, S(═O)₂C(═O)NR^{c}R^{d}, —S(═O)₂NR^{c}C(═O)NR^{c}R^{d} and —S(═O)₂(N═)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S;

X is selected from the group consisting of N, P, and S;

Q is a counter anion or is absent;

R⁵ and R⁶ are each independently selected from the group consisting of alkyl, aryl, cycloalkyl, heteroalkyl, heteroaryl, and heterocycloalkyl, each of which may be optionally substituted; or R⁵ and R⁶ together with the X atom to which they are attached form heterocycloalkyl group, which may be optionally substituted; and

R⁷ is alkyl, aryl, cycloalkyl, heteroalkyl, heteroaryl, or heterocycloalkyl, each of which may be optionally substituted, and may further be O when X is N, with the proviso that R⁷ is absent when X is S;

and with the proviso that if R is a divalent cycloalkylene radical, n will not exceed the integer 11.

In one aspect, the amides of compounds of Formula (I) as represented herein are —NRpRq amides of sulfonic acid, carboxylic acid and phosphonic acids, wherein Rp and Rq independently are selected from the group consisting of hydrogen, (C₁₋₄)alkyl and aryl.

In another aspect, the compound is a compound of Formula (II)

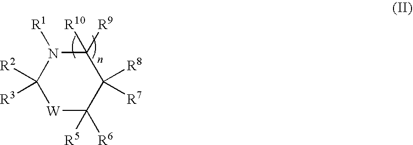

or a derivative thereof, wherein:

n is 0 or 1;

W is NR⁴, O, S, S(═O) or S(═O)₂;

R¹ is H, Cl, Br, -L-X or optionally substituted alkyl or heteroalkyl;

R² and R³ are each independently H, -L-X, or optionally substituted alkyl or heteroalkyl, or R² and R³ together with the carbon to which they are attached form a carbonyl, -L-X or an optionally substituted cycloalkyl or heterocycloalkyl group;

R⁴ is H, Cl, Br, -L-X or optionally substituted alkyl or heteroalkyl;

R⁵ and R⁶ are each independently H, -L-X or optionally substituted alkyl or heteroalkyl; or R⁵ and R⁶ together with the carbon to which they are attached form a carbonyl, -L-X or an optionally substituted cycloalkyl or heterocycloalkyl group;

R⁷ and R⁸ are each independently H, -L-X or optionally substituted alkyl or heteroalkyl; or R⁷ and R⁸ together with the carbon to which they are attached form a carbonyl, -L-X or an optionally substituted cycloalkyl or heterocycloalkyl group;

R⁹ and R¹⁰ are each independently H, -L-X or optionally substituted alkyl or heteroalkyl; or R⁹ and R¹⁰ together with the carbon to which they are attached form a carbonyl, -L-X or an optionally substituted cycloalkyl or heterocycloalkyl group;

each L is independently an optionally substituted C₁₋₆ alkyl, heteroalkyl, cycloalkyl or heterocycloalkyl group; and

each X is independently —SO₃H, —N⁺R^{a}R^{b}R^{c}, —B(OH)₂, —CO₂H, —PO₃H₂ or —PO₃HR^{a} and R^{a}, R^{b}, and/or R^{c} are independently a bond or an optionally substituted alkyl or heteroalkyl groups, or may form, together with the N to which they are attached, a heterocycloalkyl group;

with the provisos that:
at least one of R¹, R², R³, R⁴, R⁵, R⁶, R⁷, R⁸, R⁹ or R¹⁰ is -L-X; and
at least one of R² and R³, R⁵ and R⁶, or R⁷ and R⁸, together with the carbon to which they are attached, form a carbonyl; provided that (i) R⁵, R⁶ and the carbon to which they are attached, and R⁷, R⁸ and the carbon to which they are attached, are not both carbonyl; and (ii) R⁷, R⁸ and the carbon to which they are attached, and R⁹, R¹⁰ and the carbon to which they are attached, are not both carbonyl.

In certain compounds of Formula (II), n is 0. For clarity, in these compounds, R⁹ and R¹⁰ are absent.

In certain compounds of Formula (II), W is NR⁴ or O.

In certain compounds of Formula (II), R¹ and R⁴ are not both H. In certain compounds of Formula (II), at least one of either R¹ or R⁴ is independently Cl or Br.

In certain compounds of Formula (II), R¹ is Cl.

In certain compounds of Formula (II), R⁴ is Cl. In other compounds of Formula (II), R⁴ is alkyl. In yet other compounds of Formula (II), R⁴ is -L-X.

In certain compounds of Formula (II), R², R³ and the carbon to which they are attached; R⁵, R⁶ and the carbon to which they are attached; R⁷, R⁸ and the carbon to which they are attached; and/or R⁹, R¹⁰ and the carbon to which they are attached, independently form an optionally substituted cycloalkyl or heterocycloalkyl group. In such cases, the resulting compounds may be spiro compounds. For example, in certain compounds of Formula (II), R² and R³, R⁵ and R⁶, R⁷ and R⁸, and/or R⁹ and R¹⁰, and the carbon to which they are attached, can be a N,N-dimethylpyrrolidinium or N,N-dimethylpiperidinium group (in which case the compound may be referred to as a spiro compound). For clarity, in these compounds, R² and R³, R⁵ and R⁶, R⁷ and R⁸ and/or R⁹ and R¹⁰ are considered to be -L-X, as illustrated by the following nonlimiting example:

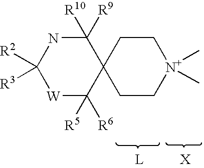

In certain compounds of Formula (II), L is a C₁₋₆ alkyl group. For example, in certain compounds, L can be —(CH₂)—, —(CH₂—CH₂)— or —(CH₂)₃—. In other compounds of Formula (II), L is a C₁₋₆ alkyl group wherein one or more of the carbon atoms is replaced with —O—, —CF₂—, —CHF—, —C(CF₃)H—, —C(═O)—, —C(═O)O—, —OC(═O)—, —C(═O)NR^{d}—, —NR^{d}C(═O)—, —P(═O)(OR^{e})O—, —OP(═O)(OR^{e})—, —P(═O)(OR^{e})NR^{f}—, —NR^{f}P(═O)(OR^{e})—, —S(═O)₂—, —S(═O)₂O—, —OS(═O)₂—, —S(═O)₂NR^{g}—, —NR^{g}S(═O)₂—, or heteroaryl; and R^{d}, R^{e}, R^{f} and R^{g} are each independently selected from the group consisting of hydrogen, alkyl, aryl, cycloalkyl, heteroalkyl, heteroaryl and heterocycloalkyl, each of which may be optionally and independently substituted.

In certain compounds of Formula (II), X is —SO₃H or —N⁺R^{a}R^{b}R^{c}.

In certain compounds of Formula (II), R^{a}, R^{b}, and R^{c} are independently optionally substituted alkyl. For example, in certain compounds of Formula (II), R^{a}, R^{b} and R^{c} are methyl. In other compounds of Formula (II), R^{a} may be alkyl (e.g. methyl) and R^{b} and R^{c} together with the N to which they are attached may form a pyrrolidinium group.

In certain compounds of Formula (II), the compound is an acid, e.g. a sulfonic acid. In other compounds of Formula (II), the compound is a salt, e.g. a pharmaceutically acceptable salt. For example, a compound of Formula (II) may be a sodium, chloride, dichloride, acetate, ammonium, or substituted or quaternary ammonium salt.

Another aspect of the current disclosure relates to compounds of Formula (III)

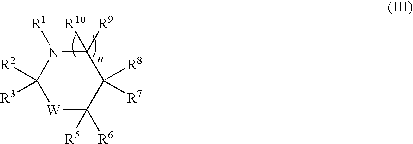

or a derivative thereof, wherein n is 0 or 1;

W is NR⁴ or O;

R¹ is H, Cl, Br, or optionally substituted alkyl;

R², R³, R⁵, R⁶, R⁷, R⁸, R⁹, and R¹⁰ are each independently H or optionally substituted alkoxy, alkyl, heteroalkyl, aryl, heteroaryl, arylalkyl, or hydroxyl; or R² and R³ together with the carbon to which they are attached, R⁵ and R⁶ together with the carbon to which they are attached, R⁷ and R⁸ together with the carbon to which they are attached, and/or R⁹ and R¹⁰ together with the carbon to which they are attached form a carbonyl or an optionally substituted cycloalkyl or heterocycloalkyl group;

R⁴ is H, Cl, Br, or optionally substituted alkyl, with the proviso that R¹ and R⁴ are not both H.

The methods described herein also comprise administering a first active agent comprising an N-halogenated or N,N-dihalogenated amine compounds described herein (e.g. compound of any of Formulae I, II or III), and a second active agent, wherein the second active agent is an antiinfective agent of a different class than the first active agent. Such second active agents include, but are not limited to, oral antifungal agents such as terbinafine, itraconazole, and fluconazole, as well as topical antifungal agents such as allylamines (e.g. terbinafine), triazoles (e.g. itraconazole and fluconazole), imidazole derivatives (e.g. ketoconazole, miconazole, clotrimazole, and econazole), amorolfine, ciclopirox, alamine, sodium pyrithione, and combinations thereof such, for example, bifonazole and urea, and propylene glycol and urea and lactic acid. The second active agent may also be an oral or topical antibacterial and antiviral agent. Suitable antibacterial agents include penicillin, ampicillin, amoxicillin, cefalexin, erythromycin ethylsuccinate, bacampicillin hydrochloride, minocycline hydrochloride, chloramphenicol, tetracycline, and erythromycin. Antibacterial agents useful for topical administration are reported, for example, in the reference Hirschmann, Arch Dermatol. 1988; 124 (11):1691-1700. Suitable antiviral agents include acyclovir, amantadine, cidofovir, fomivirsen, foscarnet, gancyclovir, zidovudine and lamivudine and protease inhibitors in combination, penciclovir and the oral prodrugs valaciclovir and famciclovir, and ribavirin.

The methods described herein also comprise administering an active agent comprising an N-halogenated or N,N-dihalogenated compounds described herein, and a penetration enhancer.

This disclosure also describes methods of treating or preventing fungal infections of the nail, claw, or hoof, comprising administering a formulation, wherein the formulation comprises (i) an N-halogenated or N,N-dihalogenated compound or an N-halogenated or N,N-dihalogenated amine compound described herein; and (ii) a polymer. The polymer can be a water-swellable polymer such as a poly(ethylene oxide) or a poly acrylic acid. The formulation can include one or more polymers, e.g. the composition can comprise a mixture of a poly(ethylene oxide) and a poly acrylic acid. In certain embodiments, the formulations may also comprise a solvent, co-solvent, humectant, film-forming agent, carrier, penetration enhancer, plasticizer, or other inactive ingredients, or a combination thereof. In certain formulations, the compound can be stabilized.

Methods described herein also comprise administering an active agent comprising an N-halogenated or N,N-dihalogenated compounds described herein in the form of a gel, cream, lotion, spray, film-forming solution, lacquer, or injectable solution. In certain embodiments, the active agent comprising an N-halogenated or N,N-dihalogenated compounds may be administered or applied to a subject with a bandage or patch. In other embodiments, the active agent of the compositions comprising the active agent may be applied with a pipette or eye-dropper, or a cotton or fabric applicator.

The details of one or more embodiments are set forth in the accompanying figures and the description below. Other features, objects, and advantages will be apparent from the description and drawings, and from the claims.

### DESCRIPTION OF DRAWINGS

FIG. 1 is a chart showing the effects of the application of test compositions described herein to a first side of human nail samples on ATP production of T. rubrum cultured on the second side of the nail samples.

### DETAILED DESCRIPTION

This application is not limited to particular methodologies (e.g., modes of administration) or the specific compositions described, as such, may vary. It is also to be understood that the terminology used herein is for the purpose of describing particular embodiments only, and is not intended to be limiting, since the scope of the present application will be limited only by the appended claims and their equivalents.

Unless defined otherwise, all technical and scientific terms used herein have the same meaning as commonly understood by one of ordinary skill in the art to which this application belongs. It must be noted that as used herein and in the appended claims, the singular forms such as “a”, “and”, and “the” include plural referents unless the context clearly dictates otherwise. Thus, e.g., reference to “the compound” includes a plurality of such compounds, and so forth. A dash at the front or end of a chemical group is a matter of convenience; chemical groups may be depicted with or without one or more terminal dashes without losing their ordinary meaning. Also, certain commonly used alternative chemical names may or may not be used. For example, a divalent group such as a divalent “alkyl” group, a divalent “aryl” group, etc., may also be referred to as an “alkylene” group or an “alkylenyl” group, an “arylene” group or an “arylenyl” group, respectively.

As utilized in accordance with the present disclosure, the following terms, unless otherwise indicated, shall be understood to have the following meanings:

“Alkyl” refers to a saturated, branched, or straight-chain monovalent hydrocarbon radical derived by the removal of one hydrogen atom from a single carbon atom of a parent alkane. Alkyl groups include, but are not limited to, methyl; ethyl; propyls such as propan-1-yl, propan-2-yl (iso-propyl), cyclopropan-1-yl, etc.; butyls such as butan-1-yl, butan-2-yl (sec-butyl), 2-methyl-propan-1-yl (iso-butyl), 2-methyl-propan-2-yl (t-butyl), cyclobutan-1-yl; pentyls; hexyls; octyls; dodecyls; octadecyls; and the like. An alkyl group comprises from 1 to about 22 carbon atoms, e.g., from 1 to 22 carbon atoms, e.g. from 1 to 12 carbon atoms, or, e.g., from 1 to 6 carbon atoms.

“Alkylcycloalkyl” refers to an alkyl radical, as defined above, attached to a cycloalkyl radical, as defined herein. Alkylcycloalkyl groups include, but are not limited to, methyl cyclopentyl, methyl cyclobutyl, ethyl cyclohexyl, and the like. An alkylcycloalkyl group comprises from 4 to about 32 carbon atoms, i.e. the alkyl group can comprise from 1 to about 22 carbon atoms and the cycloalkyl group can comprise from 3 to about 10 carbon atoms.

“Active agent” refers to a pharmaceutically active compound, for example an antifungal, antibacterial, or antiviral compound. Active agents include compounds of any of Formulae I, II or III, including derivatives thereof.

“Acyl” refers to a radical —C(═O)R, where R is hydrogen, alkyl, cycloalkyl, cycloheteroalkyl, aryl, arylalkyl, heteroalkyl, heteroaryl, or heteroarylalkyl as defined herein, each of which may be optionally substituted, as defined herein. Representative examples include, but are not limited to formyl, acetyl, cyclohexylcarbonyl, cyclohexylmethylcarbonyl, benzoyl, benzylcarbonyl and the like.

“Acylamino” (or alternatively “acylamido”) refers to a radical —NR′C(═O)R, where R′ and R are each independently hydrogen, alkyl, cycloalkyl, cycloheteroalkyl, aryl, arylalkyl, heteroalkyl, heteroaryl, or heteroarylalkyl, as defined herein, each of which may be optionally substituted, as defined herein. Representative examples include, but are not limited to, formylamino, acetylamino (i.e., acetamido), cyclohexylcarbonylamino, cyclohexylmethyl-carbonylamino, benzoylamino (i.e., benzamido), benzylcarbonylamino and the like.

“Acyloxy” refers to a radical —OC(═O)R, where R is hydrogen, alkyl, cycloalkyl, cycloheteroalkyl, aryl, arylalkyl, heteroalkyl, heteroaryl or heteroarylalkyl, as defined herein, each of which may be optionally substituted, as defined herein. Representative examples include, but are not limited to, acetyloxy (or acetoxy), butanoyloxy, benzoyloxy and the like.

“Alkoxy” refers to a radical —OR where R represents an alkyl or cycloalkyl group as defined herein, each of which may be optionally substituted, as defined herein. Representative examples include, but are not limited to, methoxy, ethoxy, propoxy, butoxy, cyclohexyloxy and the like.

“Alkoxycarbonyl” refers to a radical —C(═O)-alkoxy where alkoxy is as defined herein.

“Alkylsulfonyl” refers to a radical —S(═O)₂R where R is an alkyl or cycloalkyl group as defined herein, each of which may be optionally substituted, as defined herein. Representative examples include, but are not limited to, methylsulfonyl, ethylsulfonyl, propylsulfonyl, butylsulfonyl and the like.

“Aryl” refers to an aromatic hydrocarbon group which may be a single aromatic ring or multiple aromatic rings which are fused together, linked covalently, or linked to a common group such as a methylene or ethylene moiety. Aryl groups include, but are not limited to, groups derived from, naphthylene, anthracene, azulene, benzene, biphenyl, chrysene, cyclopentadiene, diphenylmethyl, fluoranthene, fluorene, indane, indene, naphthalene, pentalene, perylene, phenalene, phenanthrene, pyrene, triphenylene, and the like. An aryl group comprises from 6 to about 20 carbon atoms, e.g., from 6 to 20 carbon atoms, e.g. from 6 to 10 carbon atoms.

“Arylalkyl” refers to an alkyl group in which one of the hydrogen atoms bonded to a carbon atom is replaced with an aryl group. Arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl may be used. An arylalkyl group comprises from 7 to about 42 carbon atoms, e.g. the alkyl group can comprise from 1 to about 22 carbon atoms and the aryl group can comprise from 6 to about 20 carbon atoms.

“Carboxylate” refers to the group RCO₂—, where R can be hydrogen, alkyl, aryl, cycloalkyl, heteroalkyl, or heteroaryl as defined herein, each of which may be optionally substituted, as defined herein.

“Carbamoyl” refers to the radical —C(═O)N(R)₂ where each R group is independently hydrogen, alkyl, cycloalkyl or aryl as defined herein, which may be optionally substituted, as defined herein.

“Cycloalkyl” refers to a saturated cyclic alkyl radical. Typical cycloalkyl groups include, but are not limited to, groups derived from cyclopropane, cyclobutane, cyclopentane, cyclohexane, and the like. A cycloalkyl group comprises from 3 to about 10 carbon atoms, e.g. from 3 to 10 carbon atoms, or, e.g. from 3 to 6 carbon atoms.

“Derivative” refers to salts, esters (e.g. esters with C₁-C₆ alkanols), amides, prodrugs, and tautomers of compounds described herein, including salts of those esters, amides, prodrugs and tautomers. Derivatives include pharmaceutically acceptable derivatives, including pharmaceutically acceptable salts, esters and prodrugs.

“Electron-withdrawing group” refers to atoms or functional groups which are electronegative either through a resonance effect or an inductive effect. Examples of such atoms and functional groups include, but are not limited to —CO₂R⁰, —NO₂, —SO₃R⁰, —PO₃R⁰R⁰⁰, cyano, halogen (F, Cl, Br, I), and haloalkyl, where R⁰ and R⁰⁰ are independently H, alkyl, aryl, cycloalkyl, heteroalkyl, heteroaryl, or cycloheteroalkyl group, as defined herein, each of which may be optionally substituted.

“Halide” refers to a halogen bearing a negative charge, including fluoride, chloride, bromide, and iodide.

“Halo” refers to a halogen, including fluoro, chloro, bromo and iodo.

“Heteroalkyl” refer to an alkyl radical in which one or more of the carbon atoms (and any associated hydrogen atoms) are each independently replaced with the same or different heteroatomic groups. Heteroatomic groups include, but are not limited to, —NR⁰—, —O—, —S—, —PH—, —P(O)₂—, —S(O)—, —S(O)₂—, and the like, where R⁰ is defined above. Heteroalkyl groups include, but are not limited to, —O—CH₃, —CH₂—O—CH₃, —S—CH₃, —CH₂—S—CH₃, —NR⁰—CH₃, —CH₂—NR⁰⁰—CH₃, and the like, where R⁰ and R⁰⁰ are defined above. A heteroalkyl group can comprise from 1 to about 22 carbon and hetero atoms, e.g., from 1 to 22 carbon and heteroatoms, e.g. from 1 to 12 carbon and hetero atoms, e.g., from 1 to 6 carbon and hetero atoms.

“Heteroaryl” refers to an aryl group in which one or more of the carbon atoms (and any associated hydrogen atoms) are each independently replaced with the same or different heteroatomic groups. Typical heteroatomic groups include, but are not limited to, —N—, —O—, —S—, and —NR⁰—, where R⁰ is defined above. Typical heteroaryl groups include, but are not limited to, groups derived from acridine, carbazole, carboline, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. A heteroaryl group comprises from 5 to about 20 atoms, e.g., from 5 to 20 atoms, e.g. from 5 to 10 atoms.

“Heterocycloalkyl” refers to unsaturated cycloalkyl radical in which one or more carbon atoms (and any associated hydrogen atoms) are independently replaced with the same or different heteroatom. Typical heteroatoms to replace the carbon atom(s) include, but are not limited to, N, P, O, S, etc. Typical heterocycloalkyl groups include, but are not limited to, groups derived from epoxides, imidazolidine, morpholine, piperazine, piperidine, pyrazolidine, pyrrolidine, quinuclidine, and the like. The heterocycloalkyl group comprises between 3 and 10 carbon atoms.

“Hydroxy” refers to the group OH.

“Nail or nearby tissue” refers to tissues and structures of the nail or claw, including the nail plate, nail bed, nail matrix, nail root, eponychium (cuticle), perinychium, hyponychium, and proximal and lateral nail folds, and corresponding structures of the claw. This term also refers to tissues and structures of the hoof, including the coronet, wall, toe, quarter, heel, bulb, frog and sole.

“Penetrate,” “penetrates,” and “penetration” and similar terms refers the ability of a compound described herein to fully penetrate a nail, claw, or hoof, that is, to be transported from one surface of the nail (e.g. dorsal) to the other side (e.g. ventral). Penetration can be measured using an infected nail model, e.g., by infecting the ventral side of a nail with a fungus (e.g. T. mentagrophytes or T. rubrum), administering an active agent to the dorsal side of the nail, and determining the time at which fungus is killed, as determined by visual inspection or otherwise. Penetration can also be measured by detecting the active agent, e.g. by UV/Vis or other analytical instrumentation, at one side of the nail (e.g. ventral) after the active agent is administered at the other side of the nail (e.g. dorsal). An active agent may be described as one which “penetrates” a nail within a certain time, meaning that the presence of the active agent or its antifungal effect has been detected as described above within the time specified.

“Phosphate” refers to the group (R)ₙPO₄⁽³⁻ⁿ⁾⁻, where n is 0, 1 or 2 and R can be hydrogen, alkyl, aryl, cycloalkyl, heteroalkyl, or heteroaryl as defined herein, each of which may be optionally substituted.

“Pharmaceutically acceptable” refers to that which is useful in preparing a pharmaceutical composition that is generally safe, non-toxic, and neither biologically nor otherwise undesirable, and includes that which is acceptable for veterinary as well as human pharmaceutical use.

“Pharmaceutically acceptable salt” refers to a salt of a compound that is pharmaceutically acceptable and that possesses the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-naphthalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. A representative list of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, which are hereby incorporated by reference.

“Pharmaceutically acceptable carrier” refers to a pharmaceutically acceptable diluent, adjuvant, excipient or vehicle and the like with which a compound is combined for administration.

“Pharmaceutical composition” as used herein comprises one or more active agents and a pharmaceutically acceptable carrier.

“Prevent”, “preventing” and “prevention” of an infection refer to reducing the risk of a subject from developing an infection, or reducing the frequency or severity of an infection.

“Prodrug” and “prodrugs” refer to compounds that are rapidly transformed in vivo to yield a compound of the Formulae describe herein, for example by hydrolysis (chemical or enzymatic). By way of example but not limitation, one type of prodrug is esters, for example esters derived from pharmaceutically acceptable aliphatic carboxylic acids such as formats, acetates, propionates, butyrates, acrylates, ethylsuccinates, and the like. Further examples of prodrugs can be found in J. Rautio et al. Prodrugs: design and clinical applications, Nat. Rev. Drug Discov., 7, 255-270 (2008).

“Salt” refers to a cation or anion coupled with an anion or a cation respectively, either in solution or as a solid. Salts include pharmaceutically acceptable salts as well as solvent addition forms (solvates) of the same salt. Unless specified in reaction schemes, where certain compounds described herein are named or depicted as a particular salt (e.g. the chloride), all other salt forms are within the scope of this disclosure.

“Stable” or “stability” refers to the ability of a given formulation to retain a minimum concentration of N-halogenated or N,N-dihalogenated amine compound at a certain temperature or temperature range over a certain amount of time. For example, a certain formulation may have a stability of 90% for at least 90 days when stored at about 25° C., meaning that it retains at least about 90% of the initial concentration of N-halogenated or N,N-dihalogenated amine compound under those conditions.

“Sulfate” refers to the group —OSO₃H or SO₄²⁻.

“Sulfonate” refers to the group —OSO₂R, where R can be alkyl, aryl, cycloalkyl, heteroalkyl or heteroaryl, or as defined herein.

“Subject” refers to any animal having a nail, claw, or hoof, including but not limited to a bird and reptile; an ungulate such as a horse, cow, bull (and other types of cattle), goat, pig, and sheep; a dog and cat; and other mammals such as a human.

A “substituted” group refers to a group wherein from 1 to about 5 (e.g. from 1 to 5, e.g. from 1 to 3) hydrogens are replaced with a substituent such as an acyl, alkoxy, alkyl, alkoxycarbonyl, alkylsulfonyl, amino, acyloxy, aryl, carboxyl, carbamoyl, cycloalkyl, halo, heteroalkyl, heteroaryl, cycloheteroaryl, oxo, hydroxy, acylamino, electron-withdrawing group, or a combination thereof. In certain aspects, substituents include, without limitation, cyano, hydroxy, nitro, trifluoromethyl, methoxy, phenyl, and carboxyl.

In one aspect, the N-halogenated or N,N-dihalogenated amine compound may be a compound of Formula (I)

A-C(R¹R²)R(CH₂)ₙC(R³R⁴)—Y—Z (I)

or a derivative thereof, wherein

A is hydrogen, HalNH— or Hal₂N—, wherein Hal is a halogen selected from the group consisting of chloro, bromo and iodo;

R¹ is hydrogen or an optionally substituted group selected from the group consisting of alkyl, cycloalkyl, heteroalkyl, aryl, heteroaryl, and heterocycloalkyl groups, and —COOH;

R² is hydrogen or an optionally substituted group selected from the group consisting of alkyl, cycloalkyl, heteroalkyl, aryl, heteroaryl, and heterocycloalkyl groups, or R¹ and R² together with the carbon atom to which they attach form an optionally substituted cycloalkyl or heterocycloalkyl group;

R is a carbon-carbon single bond or a divalent cycloalkylene radical with three to six carbon atoms,

n is 0 or an integer from 1 to 13;

R³ and R⁴ are each independently selected from the group consisting of hydrogen, fluoro, —NHHal, NHal₂, and an optionally substituted group selected from the group consisting of alkyl, cycloalkyl, heteroalkyl, aryl, heteroaryl, and heterocycloalkyl groups;

Y is selected from a group consisting of a single bond; —O—, —CF₂—, —CHF—, —C(═O)—, —C(═O)O—, —OC(═O)—, —C(═O)NR^{a}—, —NR^{a}C(═O)—, —P(═O)(OR^{b})O—, —OP(═O)(OR^{b})—, —P(═O)(OR^{b})NR^{c}—, —NR^{c}P(═O)(OR^{b})—, —S(═O)₂, —S(═O)₂O—, —OS(═O)₂—, —S(═O)₂NR^{d}—, —NR^{d}S(═O)₂—, or heteroaryl wherein R^{a}, R^{b}, R^{c} and R^{d} are each independently selected from the group consisting of hydrogen, and optionally substituted alkyl, aryl, cycloalkyl, heteroalkyl, heteroaryl, and heterocycloalkyl; a divalent (C₁₋₁₈)alkyl group in which, optionally, one or two methylene groups are replaced with a mono- or di-substituted methylene group; and a divalent (C₁₋₁₈)heteroalkyl group wherein the divalent (C₁₋₁₈)heteroalkyl group is a divalent (C₁₋₁₈)alkyl group in which, optionally, one or two methylene groups are replaced with 1 or 2-NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂—, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, and (C₆₋₁₀)aryl(Cᵢ₋₄)alkylC(═O)—wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄) alkyl, the heterocycloalkyl group containing 2-10 carbon atoms and 1 to 4 heteroatoms selected from N, O or S;

Z is selected from the group consisting of hydrogen, —CO₂H, —CONH₂, —SO₃H, —SO₂NH₂, —P(═O)(OH)₂, —B(OH)₂, —[X(R⁵)(R⁶)R⁷]Q, —S(═O)₂NR^{c}R^{d}, —S(═O)₂NHC(═O)R^{e}, S(═O)₂C(═O)NR^{c}R^{d}, —S(═O)₂NR^{c}C(═O)NR^{c}R^{d} and —S(═O)₂(N═)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S;

X is selected from the group consisting of N, P, and S;

Q is a counter anion or is absent;

R⁵ and R⁶ are each independently selected from the group consisting of alkyl, aryl, cycloalkyl, heteroalkyl, heteroaryl, and heterocycloalkyl, each of which may be optionally substituted; or R⁵ and R⁶ together with the X atom to which they are attached form heterocycloalkyl group, which may be optionally substituted; and

R⁷ is alkyl, aryl, cycloalkyl, heteroalkyl, heteroaryl, or heterocycloalkyl, each of which may be optionally substituted, and may further be O when X is N, with the proviso that R⁷ is absent when X is S;

and with the proviso that if R is a divalent cycloalkylene radical, n will not exceed the integer 11.

In one aspect, the amides as represented herein are —NRpRq amides of sulfonic acid, carboxylic acid and phosphonic acids, wherein Rp and Rq independently are selected from the group consisting of hydrogen, (C₁₋₄)alkyl and aryl.

One aspect of the current disclosure relates compounds of Formula (IA)

A-C(R¹R²)R(CH₂)ₙ—C(R³R⁴)—Y—Z (IA)

or a derivative thereof, wherein

A is hydrogen, Hal₂N—, or HalHN, wherein Hal is halogen selected from the group consisting of chloro, bromo and iodo;

R¹ is hydrogen, (C₁₋₆)alkyl or the group —COOH;

R² is hydrogen or (C₁₋₆)alkyl, or R¹ and R² together with the carbon atom to which they attach form a (C₃₋₆)cycloalkyl ring;

R is a carbon-carbon single bond or a divalent cycloalkylene radical with three to six carbon atoms;

n is 0 or an integer from 1 to 13,

R³ is hydrogen, (C₁₋₆)alkyl, —NHHal, or —NHal₂;

R⁴ is hydrogen or (C₁₋₆)alkyl;

Y is a single bond;

and Z is selected from the group consisting of hydrogen, —CO₂H, —CONH₂, —SO₃H, —SO₂NH₂, —P(═O)(OH)₂ and —B(OH)₂.

Within this aspect, if R is a divalent cycloalkylene radical, n will not exceed the integer 11. That is, n may be 0, 1, 2, 3, 4, 5, 6, 7, 8, 9, 10 or 11. In other words, an amine compound including an Z acidic group will have up to 16 chain atoms. In the divalent cycloalkylene radical or in the divalent radical —(CH₂)ₙ— one hydrogen may be substituted with —NHal₂. Compounds of Formula (IA) may contain up to a total of three —NHal₂ or —NHHal groups, for example, one or two —NHal₂ or —NHHal groups. In certain aspects, compounds of Formula (IA) contain one —NHal₂ group, which may be in the alpha-, beta-, gamma-, delta-, epsilon- or omega-position of an acidic R¹ (if R¹ is —COOH) or Z group.

Another aspect of the current disclosure relates to compounds of Formula (IB)

A-C(R¹R²)—C(R³R⁴)—Y—Z (IB)

or derivative thereof, wherein

A is selected from the group consisting of hydrogen, Hal₂N—, and HalHN;

Hal is halogen selected from the group consisting of chloro and bromo;

R¹ and R² are each independently selected from the group consisting of (C₁₋₅)alkyl, heteroalkyl, halo(C₁₋₅)alkyl, (C₃₋₆)cycloalkyl, (C₃₋₆)cycloalkyl(C₁₋₃)alkyl, (C₆₋₁₀)aryl(C₁₋₄)alkyl, (C₆₋₁₄)aryl, heteroaryl, and (C₃₋₁₀)heterocycloalkyl, or R₁ and R₂ together with the carbon atom to which they are attached to form a (C₃₋₁₂)cycloalkyl or (C₃₋₁₂)heterocycloalkyl;

R³ and R⁴ are each independently selected from the group consisting of hydrogen, fluoro, (C₁₋₅)alkyl, heteroalkyl, halo(C₁₋₅)alkyl, (C₃₋₆)cycloalkyl, (C₃₋₆)cycloalkyl(C₁₋₃)alkyl, (C₆₋₁₀)aryl(C₁₋₄)alkyl, (C₆₋₁₄)aryl, heteroaryl, and (C₃₋₁₀) heterocycloalkyl, or R³ and R⁴ together with the carbon atom to which they are attached to form a (C₃₋₁₂)cycloalkyl, or (C₃₋₁₂) heterocycloalkyl;

Y is selected from a group consisting of single bond, —O—, a divalent (C₁₋₁₈)alkyl group in which optionally one or two methylene groups are replaced with a mono- or di-substituted methylene group, and a (C₁₋₁₈)heteroalkyl group,

with the proviso that when R¹ is (C₁₋₅)alkyl or when R¹ and R² together with the carbon atom to which they attach form a (C₃₋₆)cycloalkyl, then Y must be —O— or a divalent (C₁₋₁₈) alkyl group wherein one or two methylene groups are replaced with a substituted methylene group or Y must be a divalent (C₁₋₁₈) heteroalkyl group wherein the (C₁₋₁₈) heteroalkyl group is a (C₁₋₁₈)alkyl group where one or two methylene groups are replaced with a by —NR′—, —O—, —S—, —S(═O)— or —S(═O)₂—;

R′ is hydrogen or is selected from the group consisting of Cl, Br, (C₁₋₅)alkyl, (C₃₋₆)cycloalkyl, (C₆₋₁₀)aryl(C₁₋₄)alkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄) alkyl, the heterocycloalkyl group containing 2-10 carbon atoms and 1 to 4 heteroatoms selected from N, O or S; and

Z is selected from the group consisting of hydrogen, —CO₂H, —CONH₂, —SO₃H, —SO₂NH₂, —P(═O)(OH)₂ and —B(OH)₂.

Another aspect of the current disclosure relates to compounds of Formula (IB), wherein R¹ and R² together with the carbon atoms to which they are attached form a ring system with 4 to 7 carbon ring members, wherein optionally one or two ring members are nitrogen.

Another aspect of the current disclosure relates to compounds of Formula (IC)

A-C(R¹R²)(CH₂)ₙY(CH₂)ₘ—Z (IC)

or a derivative thereof, wherein:

A is HalHN— or Hal₂N—, wherein Hal is halogen selected from the group consisting of chloro and bromo;

R¹ and R² are each independently selected from the group consisting of alkyl, aryl, cycloalkyl, heteroalkyl, heteroaryl, and heterocycloalkyl, each of which may be optionally substituted; or R¹ and R² together with the carbon atom to which they are attached form a cycloalkyl or heterocycloalkyl group, each of which may be optionally substituted;

Y is selected from the group consisting of a single bond, —O—, —CF₂—, —CHF—, —C(═O)—, —C(═O)O—, —OC(═O)—, —C(═O)NR^{a}—, —NR^{a}C(═O)—, —P(═O)(OR^{b})O—, —OP(═O)(OR^{b})—, —P(═O)(OR^{b})NR^{c}—, —NR^{c}P(═O)(OR^{b})—, —S(═O)₂, —S(═O)₂O—, —OS(═O)₂—, —S(═O)₂NR^{d}—, —NR^{d}S(═O)₂—, or heteroaryl, wherein R^{a}, R^{b}, R^{c} and R^{d} are each independently selected from the group consisting of hydrogen, and optionally substituted alkyl, aryl, cycloalkyl, heteroalkyl, heteroaryl, and heterocycloalkyl;

Z is —[X(R⁵)(R⁶)R⁷]Q, wherein Q, R⁵, R⁶, and R⁷ are defined as in Formula (I) above;

n is 0 or is an integer from 1 to 12; and

m is an integer from 1 to 12.

Another aspect of the current disclosure relates to compounds of Formula (ID)

A-C(R₁R₂)(CH₂)ₙC(R³R⁴)—Z (ID)

or a derivative thereof, wherein:

A is hydrogen, HalNH— or Hal₂N—, wherein Hal is halogen selected from the group consisting of chloro, bromo and iodo;

R¹ and R² are each independently selected from an optionally substituted group selected from the group consisting of alkyl, cycloalkyl, heteroalkyl, heteroaryl, and heterocycloalkyl groups, or R¹ and R² together with the carbon atom to which they attach form a (C₃₋₆)cycloalkyl ring;

n is 0 or an integer from 1 to 13

R³ and R⁴ are independently selected from the group consisting of hydrogen, fluoro, and an optionally substituted group selected from the group consisting of alkyl, cycloalkyl, heteroalkyl, heteroaryl, and heterocycloalkyl groups;

Z is selected from the group consisting of, —SO₃H, —SO₂NH₂, —P(═O)(OH)₂, —B(OH)₂, and —[X(R⁵)(R⁶)R⁷]Q, R⁵, R⁶, and R⁷ is defined as in Formula (I) above; and

n is 0 or an integer from 1 to 6.

Another aspect of the current disclosure relates to compounds of Formula (II)

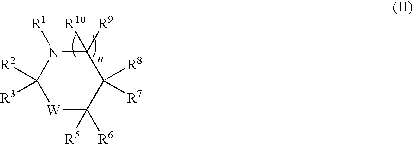

or a derivative thereof, wherein:

n is 0 or 1;

W is NR⁴, O, S, S(═O) or S(═O)₂;

R¹ is H, Cl, Br, -L-X or optionally substituted alkyl or heteroalkyl;

R² and R³ are each independently H, -L-X, or optionally substituted alkyl or heteroalkyl, or R² and R³ together with the carbon to which they are attached form a carbonyl, -L-X or an optionally substituted cycloalkyl or heterocycloalkyl group;

R⁴ is H, Cl, Br, -L-X or optionally substituted alkyl or heteroalkyl;

R⁵ and R⁶ are each independently H, -L-X or optionally substituted alkyl or heteroalkyl; or R⁵ and R⁶ together with the carbon to which they are attached form a carbonyl, -L-X or an optionally substituted cycloalkyl or heterocycloalkyl group;

R⁷ and R⁸ are each independently H, -L-X or optionally substituted alkyl or heteroalkyl; or R⁷ and R⁸ together with the carbon to which they are attached form a carbonyl, -L-X or an optionally substituted cycloalkyl or heterocycloalkyl group;

R⁹ and R¹⁰ are each independently H, -L-X or optionally substituted alkyl or heteroalkyl; or R⁹ and R¹⁰ together with the carbon to which they are attached form a carbonyl, -L-X or an optionally substituted cycloalkyl or heterocycloalkyl group;

each L is independently an optionally substituted C₁₋₆ alkyl, heteroalkyl, cycloalkyl or heterocycloalkyl group; and

each X is independently —SO₃H, —N⁺R^{a}R^{b}R^{c}, —B(OH)₂, —CO₂H, —PO₃H₂ or —PO₃HR^{a} and R^{a}, R^{b}, and/or R^{c} are independently a bond or an optionally substituted alkyl or heteroalkyl groups, or may form, together with the N to which they are attached, a heterocycloalkyl group;

with the provisos that:
at least one of R¹, R², R³, R⁴, R⁵, R⁶, R⁷, R⁸, R⁹ or R¹⁰ is -L-X; and
at least one of R² and R³, R⁵ and R⁶, or R⁷ and R⁸, together with the carbon to which they are attached, form a carbonyl; provided that (i) R⁵, R⁶ and the carbon to which they are attached, and R⁷, R⁸ and the carbon to which they are attached, are not both carbonyl; and (ii) R⁷, R⁸ and the carbon to which they are attached, and R⁹, R¹⁰ and the carbon to which they are attached, are not both carbonyl.

In certain compounds of Formula (II), n is 0. For clarity, in these compounds, R⁹ and R¹⁰ are absent.

In certain compounds of Formula (II), W is NR⁴ or O.

In certain compounds of Formula (II), R¹ and R⁴ are not both H. In certain compounds of Formula (II), at least one of either R¹ or R⁴ is independently Cl or Br.

In certain compounds of Formula (II), R¹ is Cl.

In certain compounds of Formula (II), R⁴ is Cl. In other compounds of Formula (II), R⁴ is alkyl. In yet other compounds of Formula (II), R⁴ is -L-X.

In certain compounds of Formula (II), R², R³ and the carbon to which they are attached; R⁵, R⁶ and the carbon to which they are attached; R⁷, R⁸ and the carbon to which they are attached; and/or R⁹, R¹⁰ and the carbon to which they are attached, independently form an optionally substituted cycloalkyl or heterocycloalkyl group. In such cases, the resulting compounds may be spiro compounds. For example, in certain compounds of Formula (II), R² and R³, R⁵ and R⁶, R⁷ and R⁸, and/or R⁹ and R¹⁰, and the carbon to which they are attached, can be a N,N-dimethylpyrrolidinium or N,N-dimethylpiperidinium group (in which case the compound may be referred to as a spiro compound). For clarity, in these compounds, R² and R³, R⁵ and R⁶, R⁷ and R⁸ and/or R⁹ and R¹⁰ are considered to be -L-X, as illustrated by the following nonlimiting example:

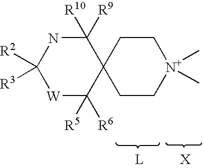

In certain compounds of Formula (II), L is a C₁₋₆ alkyl group. For example, in certain compounds, L can be —(CH₂)—, —(CH₂—CH₂)— or —(CH₂)₃—. In other compounds of Formula (II), L is a C₁₋₆ alkyl group wherein one or more of the carbon atoms is replaced with —O—, —CF₂—, —CHF—, —C(CF₃)H—, —C(═O)—, —C(═O)O—, —OC(═O)—, —C(═O)NR^{d}—, —NR^{d}C(═O)—, —P(═O)(OR^{e})O—, —OP(═O)(OR^{e})—, —P(═O)(OR^{e})NR^{f}—, —NR^{f}P(═O)(OR^{e})—, —S(═O)₂—, —S(═O)₂O—, —OS(═O)₂—, —S(═O)₂NR^{g}—, —NR^{g}S(═O)₂—, or heteroaryl; and R^{d}, R^{e}, R^{f} and R^{g} are each independently selected from the group consisting of hydrogen, alkyl, aryl, cycloalkyl, heteroalkyl, heteroaryl and heterocycloalkyl, each of which may be optionally and independently substituted.

In certain compounds of Formula (II), X is —SO₃H or —N⁺R^{a}R^{b}R^{c}.

In certain compounds of Formula (II), R^{a}, R^{b}, and R^{c} are independently optionally substituted alkyl. For example, in certain compounds of Formula (II), R^{a}, R^{b} and R^{c} are methyl. In other compounds of Formula (II), R^{a} may be alkyl (e.g. methyl) and R^{b} and R^{c} together with the N to which they are attached may form a pyrrolidinium group.

In certain compounds of Formula (II), the compound is an acid, e.g. a sulfonic acid. In other compounds of Formula (II), the compound is a salt, e.g. a pharmaceutically acceptable salt. For example, a compound of Formula (II) may be a sodium, chloride, dichloride, acetate, ammonium, or substituted or quaternary ammonium salt.

Another aspect of the current disclosure relates to compounds of Formula (III)

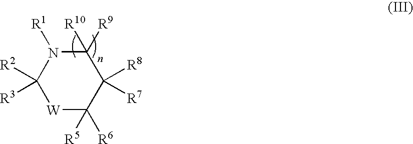

or a derivative thereof, wherein

n is 0 or 1;

W is NR⁴ or O;

R¹ is H, Cl, Br, or optionally substituted alkyl;

R², R³, R⁵, R⁶, R⁷, R⁸, R⁹, and R¹⁰ are each independently H or optionally substituted alkoxy, alkyl, heteroalkyl, aryl, heteroaryl, arylalkyl, or hydroxyl; or R² and R³ together with the carbon to which they are attached, R⁵ and R⁶ together with the carbon to which they are attached, R⁷ and R⁸ together with the carbon to which they are attached, and/or R⁹ and R¹⁰ together with the carbon to which they are attached form a carbonyl or an optionally substituted cycloalkyl or heterocycloalkyl group;

R⁴ is H, Cl, Br, or optionally substituted alkyl, with the proviso that R¹ and R⁴ are not both H.

The above-described compounds include the following compounds or a derivative thereof:
N,N-dichlorotaurine;
N,N-dichloro-2-methyltaurine;
N,N-dichloro-2,2,3,3-tetramethyl-β-alanine;
N,N-dichloro-2,2-dimethyltaurine;
N,N-dichloro-1,1,2,2-tetramethyltaurine;
N,N-dibromo-2,2-dimethyltaurine;
N,N-dibromo-1,1,2,2-tetramethyltaurine;
N,N-diiodotaurine;
N,N-dichloro-3,3-dimethylhomotaurine;
N,N-dichloro-2-methyl-2-amino-ethanesulfonic acid; and
N,N-dichloro-1-methyl-ethanesulfonic acid,
N,N-dichloro amino-trimethylene phosphonic acid;
N,N-dibromo-2-amino-5-phosphonopantanoic acid;
N,N-dichloro amino-ethylphosphonic acid diesters, such as the diethylester;
N,N-dichloro-1-amino-1-methylethane phosphonic acid;
N,N-dichloro-1-amino-2-methylethane phosphonic acid;
N,N-dichloro-1-amino-2-methylpropane phosphonic acid;
N,N-dichloro-leucine phosphonic acid;
N,N-dichloro-4-amino-4-phosphonobutyric acid;
(±) N,N-dichloro-2-amino-5-phosphonovaleric acid;
N,N-dichloro-(+)-2-amino-5-phosphonovaleric acid;
N,N-dichloro d,1-2-amino-3-phosphonopropionic acid;
N,N-dichloro-2-amino-8-phosphonooctanoic acid;
N,N-dichloro-leucine boronic acid;
N,N-dichloro-β-alanine boronic acid;
N-chlorotaurine;
N-chloro-2-methyltaurine;
N-chloro-2,2,3,3-tetramethyl-β-alanine;
N-chloro-2,2-dimethyltaurine;
N-chloro-1,1,2,2-tetramethyltaurine;
N-bromo-2,2-dimethyltaurine;
N-bromo-1,1,2,2-tetramethyltaurine;
N-iodotaurine;
N-chloro-3,3-dimethylhomotaurine;
N-chloro-2-methyl-2-amino-ethanesulfonic acid; and
N-chloro-1-methyl-ethanesulfonic acid,
N-chloro amino-trimethylene phosphonic acid;
N-bromo-2-amino-5-phosphonopantanoic acid;
N-chloro amino-ethylphosphonic acid diesters;
N-chloro amino-ethylphosphonic acid dimethylester;
N-chloro amino-ethylphosphonic acid diethylester;
N-chloro-1-amino-1-methylethane phosphonic acid;
N-chloro-1-amino-2-methylethane phosphonic acid;
N-chloro-1-amino-2-methylpropane phosphonic acid;
N-chloro-leucine phosphonic acid;
N-chloro-4-amino-4-phosphonobutyric acid;
(±) N-chloro-2-amino-5-phosphonovaleric acid;
N-chloro-(+)-2-amino-5-phosphonovaleric acid;
N-chloro d,1-2-amino-3-phosphonopropionic acid;
N-chloro-2-amino-8-phosphonooctanoic acid;
N-chloro-leucine boronic acid;
N-chloro-β-alanine boronic acid;
(1-(dichloroamino)cyclohexyl)methanesulfonic acid;
(1-(chloroamino)cyclohexyl)methanesulfonic acid;
2-(chloroamino)-N,N,N-2-tetramethylpropan-1-ammonium chloride;
2-(dichloroamino)-N,N,N-2-tetramethylpropan-1-ammonium chloride;
3-(chloroamino)-N,N,N-3-tetramethylbutan-1-ammonium chloride;
3-(dichloroamino)-N,N,N-3-tetramethylbutan-1-ammonium chloride;
3-(chloroamino)-N,N,N-triethyl-3-methylbutan-1-aminium chloride;
3-(dichloroamino)-N,N,N-triethyl-3-methylbutan-1-aminium chloride;
1-(2-(dichloroamino)-2-methylpropyl)-1-methylpiperidinium chloride;
1-(2-(chloroamino)-2-methylpropyl)-1-methylpiperidinium chloride;
(2-(dichloroamino)-2-methylpropyl)dimethylsulfonium chloride;
(2-(chloroamino)-2-methylpropyl)dimethylsulfonium chloride;
(4-(dichloroamino)-4-methylpentyl)trimethylphosphonium chloride;
(4-(chloroamino)-4-methylpentyl)trimethylphosphonium chloride;
3-(3-(dichloroamino)-3-methylbutylsulfonyl)-N,N,N-trimethylpropan-1-aminium chloride;
3-(3-(chloroamino)-3-methylbutylsulfonyl)-N,N,N-trimethylpropan-1-aminium chloride;
2-(3-(dichloroamino)-3-methylbutylsulfonyl)-N,N,N-trimethylethanaminium chloride;
2-(3-(chloroamino)-3-methylbutylsulfonyl)-N,N,N-trimethylethanaminium chloride;
1-(3-(chloroamino)-3-methylbutyl)-4,4-difluoro-1-methylpiperidinium chloride;
1-(3-(dichloroamino)-3-methylbutyl)-4,4-difluoro-1-methylpiperidinium chloride;
1-(3-chloro-4-methyl-2-oxooxazolidin-4-yl)-N,N,N-trimethylmethanaminium chloride;
(3-chloro-4-methyl-2-oxooxazolidin-4-yl)methanesulfonic acid;
(3-chloro-5-methyl-2-oxooxazolidin-5-yl)methanesulfonic acid;
4-(3-(chloroamino)-3-methylbutylsulfonyl)butanoic acid;
4-(3-(dichloroamino)-3-methylbutylsulfonyl)butanoic acid;
3-(3-(chloroamino)-3-methylbutylsulfonyl)propylphosphonic acid;
3-(3-(dichloroamino)-3-methylbutylsulfonyl)propylphosphonic acid;
(3-chloro-4,4-dimethyl-2-oxooxazolidin-5-yl)methanesulfonic acid;
2-(3-chloro-4,4-dimethyl-2,5-dioxoimidazolidin-1-yl)ethanesulfonic acid;
1-chloro-2,2,5,5-tetramethylimidazolidin-4-one;
1,3-dichloro-2,2,5,5-tetramethylimidazolidin-4-one;
1-bromo-3-chloro-2,2,5,5-tetramethylimidazolidin-4-one;
1,3-dibromo-2,2,5,5-tetramethylimidazolidin-4-one;
1,3-dichloro-2,5-bis(pentamethylene)imidazolidin-4-one;
1,3-dichloro-2-pentamethylene-5,5-dimethylimidazolidin-4-one;
1,3-dichloro-2,2-dimethyl-5-pentamethyleneimidazolidin-4-one;
1,3-dichloro-2,2,5-trimethyl-5-ethylimidazolidin-4-one;
1,3-dichloro-2-hydroxy-2,5,5-trimethylimidazolidin-4-one;
3-chloro-4,4-dimethyl-2-oxazolidinone;
3-chloro-4-ethyl-4-methyl-2-oxazolidinone; and
3-chloro-5,5-dimethyl-2-oxazolidinone.

It will be appreciated that the common name “taurine” refers to “2-aminoethanesulfonic acid,” and that compounds referred to herein containing “taurine” contain this chemical motif. For instance, “N,N-dichlorotaurine” may also be referred to as “2-(dichloroamino)-ethanesulfonic acid” and “N,N-dichloro-2,2-dimethyltaurine” may also be referred to as “2-(dichloroamino)-2-methylpropanesulfonic acid.”

The compounds described above may be neutral, cationic, or in a salt form. The compounds may be identified either by their chemical structure and/or chemical name. If the chemical structure and chemical name conflict, the chemical structure is determinative of the identity of the compound. The compounds may contain one or more chiral centers and/or double bonds and therefore, may exist as stereoisomers, such as double-bond isomers (i.e., geometric isomers), enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. Compounds may exist in unsolvated forms as well as solvated forms, including hydrated forms and as N-oxides.

Suitable salts include the following: (1) acid addition salts, formed with inorganic acids such as hydrochloric acid, hydrobromic acid, sulfuric acid, nitric acid, phosphoric acid, and the like; or formed with organic acids such as acetic acid, butyric acid, propionic acid, hexanoic acid, cyclopentanepropionic acid, glycolic acid, pyruvic acid, lactic acid, valeric acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like, made by conventional chemical means; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like, made by conventional chemical means.

Examples of acid addition salts include, but are not limited to, mineral or organic acid salts of basic residues such as substituted amides (for example, when —C(═O)NH— is present) or alkali or organic salts of acidic residues (for example, when —OP(═O)(OH)— is present). Pharmaceutically acceptable salts include, but are not limited to, hydrohalides, sulfates, methosulfates (quaternary ammonium sulfates), methanesulfonates, toluenesulfonates, nitrates, phosphates, maleates, acetates, lactates, oxalates, fumerates, succinates, and the like. The pharmaceutically acceptable acid addition salts further include salts with hydrochloric, sulfonic, phosphoric, nitric acid, acetic, benzenesulfonic, toluenesulfonic, methanesulfonic acid, camphorsulfonic acid, oxalic acid, succinic acid, fumeric acid and other acids.

Lists of suitable salts are found, for example, in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, which are hereby incorporated by reference.

The current disclosure relates to methods for treating or preventing infections of the nail, claw, or hoof, comprising administering an active agent comprising an N-halogenated or N,N-dihalogenated compound. In various embodiments, the compound can be any compound of Formulae (I), (II), or (III), including compounds of the Formulae IA to ID. In certain embodiments, the infection can be a fungal infection. For example, the infection may be an infection by a dermatophyte such as Trichophyton (“T.”) rubrum, T. interdigitale (also referred to as T. mentagrophytes), Epidermophyton floccosum, T. violaceum, Microsporum gypseum, T. tonsurans, T. soudanense, T. verrucosum. In certain embodiments, the infection may be a bacterial or viral infection (either with or without a fungal infection).

The current disclosure also relates to methods for treating or preventing infections of the nail, claw, or hoof, comprising administering to a subject in need thereof a composition or formulation comprising (i) an N-halogenated or N,N-dihalogenated compound; and (ii) a polymer. In certain embodiments, a water-swellable polymer (that is, a polymer that hydrates in water to form viscous solutions or suspensions) may be used.

Examples of water-swellable polymers include poly (ethylene oxide) polymers (e.g. Polyox®) and acrylic acid polymers (e.g. Carbopol®). Commercial grades of Polyox® include but not limited to: WSR N-10, WSR N-80, WSR N-750, WSR N-3000, WSR-205, WSR-1105, WSR N-12K, WSR N-60K, WSR-301, WSR Coagulant, WSR-308 UCARFLOC Polymer 300, UCARFLOC Polymer 302, UCARFLOC Polymer 304 and UCARFLOC Polymer 309 available from Dow Chemical Company. Carbopol® homopolyers are polymers of acrylic acid crosslinked with allyl sucrose or allyl pentaerythritol. Carbopol® copolymers are polymers of acrylic acid and C10-C30 alkyl acrylate crosslinked with allyl pentaerythritol. Carbopol® interpolymers are a carbomer homopolymer or copolymer that contains a block copolymer of polyethylene glycol and a long chain alkyl acid ester. Commercial grades of carbopol include but not limited to: Carbopol® homopolymers, Carbopol® copolymers, Carbopol® interpolymers, Noveon® AA-1 Polycarbophil (“AA1” or “AA gel”), Noveon® CA-1 Polycarbophil (calcium neutralized), Noveon® CA-2 Polycarbophil (calcium neutralized) available from Noveon, Inc. (Cleveland, Ohio, USA), and other commercially available grades of Carbopol® and Polycarbophil.

The formulations may be made by mixing the N-halogenated or N,N-dihalogenated amine compounds with the polymer using water. Water/oil emulsions, hydrating agents, wetting agents, surfactants, and the like may also be used. Concentrations of polymer (e.g. Polyox® and Carbopol® of all types), may be from about 0.01% to about 75.0% (by weight) in water (or water/oil). By way example but not limitation, for direct topical application to the nail, claw, or hoof, a polymer concentration from about 0.01% to about 10%, e.g. about 0.1% to about 5%, may be used, whereas for methods using a patch or bandage, a polymer concentration of up to about 75% may be used.

Concentrations of N-halogenated or N,N-dihalogenated amine compound may range from about 0.01% to about 20.0% (by weight). For example, in certain embodiments, the concentration of N-halogenated or N,N-dihalogenated amine compound may range from about 0.1% to about 5.0% (by weight). Combinations of more than one N-halogenated or N,N-dihalogenated amine compounds may be used in certain formulations.

The formulations described herein are generally prepared as follows: a suitable polymer was hydrated slowly in purified water with or without common pharmaceutical excipients such as sodium chloride, salts and buffers. An N-halogenated or N,N-dihalogenated amine compound is then added. The solution is then mixed and adjusted to a pH between about 3.0 and about 9.0 using a suitable acid or base, e.g. NaOH and HCl.

The current disclosure also relates to methods for treating or preventing infections of the nail, claw, or hoof, comprising administering to a subject in need thereof a composition or formulation comprising (i) an first active agent comprising an N-halogenated or N,N-dihalogenated amine compound; and (ii) a second agent selected from the group consisting of a carrier, excipient, film-forming agent, humectant, penetration enhancer, plasticizer, solvent, co-solvent, plasticizer, and surfactant. A combination of second agents described above may also be used.

Suitable solvents and co-solvents include ethanol, acetone, methylacetate, ethyl acetate, butyl acetate, alkylmethyl sulfoxides (e.g. dimethyl sulfoxide), 2-propanol, methyl isobutyl ketone, 1-butanol, dichloromethane, and mixtures thereof. For example, a volatile organic solvent may be used so that a dry film containing the active agent forms over the nail after administration of the formulation. Formulations may also include pharmaceutically acceptable excipients which can be found in Remington: The Science and Practice of Pharmacy, R. Hendrickson, ed., 21st edition, Lippincott, Williams & Wilkins, Philadelphia, Pa., (2005) at pages 317-318, which is hereby incorporated herein by reference.

The penetration enhancers of the compositions and methods described herein enhance the penetration of the active agent into the nail and nearby tissue. Penetration enhancers include inorganic bases such as NaOH, ethanol, propylene glycol, glycerol, ethyl laurate, isopropyl palmitate, isopropyl myristate, laurocapram (AZONE), dioxolanes, macrocyclic ketones, 1-decyl-thioethyl-2-pyrrolidone (HP-101), oxazolidones and biodegradable penetration enhancers such as alkyl-2-(N,N-disubstituted amino) alkanoates (e.g., dodecyl-2-(N,N-dimethylamino) isopropionate (DDAIP)), N,N-disubstituted amino alkanol alkanoates) and mixtures thereof.

The penetration enhancer is present in an amount sufficient to enhance the penetration of the compounds of the present application through nail and the surrounding skin. The amount of the penetration enhancer varies necessarily according to the desired release rate and the specific antifungal agent used. Generally, the penetration enhancer is present in an amount ranging from about 0.1 weight percent to about 25 weight percent, based on the total weight of the antifungal nail coat composition. In other aspects, the penetration enhancer is present in an amount ranging from about 0.1 weight percent to about 10 weight percent, and in an amount ranging from about 0.5 weight percent to about 5 weight percent of the antifungal nail coat composition.

In general, suitable penetration enhancers include the following classes of compounds: aliphatic and aromatic alcohols, sulfoxides, fatty alcohols, fatty acids, fatty acid esters, polyols, amides, surfactants, terpenes, alkanones, organic acids and mixtures thereof.

Suitable alcohols include, without limitation, ethanol, propanol, butanol, pentanol, hexanol, octanol, nonanol, decanol, 2-butanol, 2-pentanol, benzyl alcohol, phenoxyethanol, caprylic alcohol, decyl alcohol, lauryl alcohol, 2-lauryl alcohol, myristyl alcohol, cetyl alcohol, stearyl alcohol, oleyl alcohol, linolyl alcohol, linolenyl alcohol and mixtures thereof.

Volatile aliphatic alcohols having 2 to about 5 carbon atoms can provide a dual function of serving both as volatile carrier solvent and penetration enhancer. Aromatic alcohols, such as benzyl alcohol, phenoxyethanol, and the like can provide a dual function of serving both as a substantially non-volatile, permeation enhancer and auxiliary anti-infective. In certain aspects, suitable penetration enhancers are ethanol and benzyl alcohol.

Suitable sulfoxides include dimethylsulfoxide (DMSO), decylmethylsulfoxide, and mixtures thereof.

Suitable fatty acids include valeric, heptanoic, pelargonic, caproic, capric, lauric, myristic, stearic, oleic, linoleic, linolenic, caprylic, isovaleric, neopentanoic, neoheptanoic, neononanoic, trimethyl hexanoic, neodecanoic and isostearic acids, and mixtures thereof.

Suitable fatty acid esters include isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate, ethyl laurate, sucrose monolaurate, and mixtures thereof. Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol, and mixtures thereof.

Suitable amides include urea, dimethylacetamide, diethyltoluamide, dimethylformamide, dimethyloctamide, dimethyldecamide, pyrrolidone derivatives, 1-alkyl-4-imidazolin-2-one, cyclic amides, hexamethylenelauramide and its derivatives and mixtures thereof.

Suitable pyrrolidone derivatives include 1-methyl-2-pyrrolidone, 2-pyrrolidone, 1-lauryl-2-pyrrolidone, 1-lauryl-4-carboxy-2-pyrrolidone, 1-methyl-4-carboxy-2-pyrrolidone, 1-hexyl-4-carboxy-2-pyrrolidone, 1-decylthioethyl-2-pyrrolidone (HP-101), N-cyclohexylpyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-dimethylaminopropylpyrrolidone, N-cocoylpyrrolidone, N-tallowylpyrrolidone, fatty acid esters of N-(2-hydroxymethyl)-2-pyrrolidone, and mixtures thereof.

Suitable cyclic amides include, 1-dodecylazacycloheptan-2-one (laurocapram, AZONE), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyloctyl)azacycloheptan-2-one, 1-geranylazacyclohexan-2-one, 1-geranylazacyclopentan-2,5-dione, 1-farnesylazacyclopentan-2-one, and mixtures thereof.

Suitable surfactants include anionic surfactants, cationic surfactants, nonionic surfactants, amphoteric surfactants, bile salts and lecithin.

Suitable anionic surfactants include sodium laurate, sodium lauryl sulfate, and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof.

Suitable nonionic surfactants include alpha-hydro-omega-hydroxypoly(oxyethylene)-poly(oxypropyl) poly(oxyethylene) block copolymers, polyoxyethylene ethers, polyoxyethylene sorbitan esters, polyethylene glycol esters of fatty alcohols, and mixtures thereof. Suitable alpha-hydro-omega-hydroxy-poly(oxyethylene)-poly(oxypropyl) poly(oxyethylene) block copolymers include Poloxamers 182, 184, 231, and mixtures thereof. Suitable polyoxyethylene ethers include PEG-4 lauryl ether (BRIJ 30), PEG-2 oleyl ether (BRIJ 93), PEG-10 oleyl ether (BRIJ 96), PEG-20 oleyl ether (BRIJ 99), and mixtures thereof. Suitable polyoxyethylene sorbitan esters include the monolaurate (TWEEN 20) the monopalmitate (TWEEN 40), the monostearate (TWEEN 60), the monooleate (TWEEN 80), and mixtures thereof. Suitable polyethylene glycol esters of fatty acids include polyoxyethylene (8) monostearate (MYRJ 45), polyoxyethylene (30) monostearate (MYRJ 51), the polyoxyethylene (40) monostearate (MYRJ 52), and mixtures thereof. Saccharide surfactants such as dodecylmaltoside may also be used.

Suitable amphoteric surfactants include, without limitation thereto, lauramidopropyl betaine, cocamidopropyl betaine, lauryl betaine, cocobetaine, cocamidopropylhydroxysultaine, aminopropyl laurylglutamide, sodium cocoamphoacetate, sodium lauroamphoacetate, disodium lauroamphodiacetate, disodium cocoamphodiacetate, sodium cocoamphopropionate, disodium lauroamphodipropionate, disodium cocoamphodipropionate, sodium lauriminodipropionate, disodium cocoamphocarboxymethylhydroxypropylsulfate, and the like.

Suitable bile salts include sodium cholate, sodium salts of laurocholic, glycolic and deoxycholic acids, and mixtures thereof.

Suitable terpenes include D-limonene, alpha-pinene, beta-enrene, alpha-terpineol, terpinen-4-ol, carvol, carvone, pulegone, piperitone, menthone, menthol, geraniol, cyclohexene oxide, limonene oxide, alpha-pinene oxide, cyclopentene oxide, 1,8-cineole, ylang ylang oil, anise oil, chenopodium oil, eucalyptus oil, and mixtures thereof.

Suitable organic acids include citric acid, succinic acid, salicylic acid, salicylates (including the methyl, ethyl and propyl glycol derivatives), tartaric acid, and mixtures thereof.

A penetration enhancer may also comprise an N,N-di(C₁-C₈) alkylamino substituted, (C₄-C₁₈) alkyl (C₂-C₁₈) carboxylic ester or pharmaceutically acceptable acid addition salt thereof. As used herein, the term (C₄-C₁₈) alkyl (C₂-C₁₈) carboxylic ester means an ester of a (C₄-C₁₈) alcohol and a (C₂-C₁₈) carboxylic acid. The term N,N-di(C₁-C₈) alkylamino substituted, in reference to a (C₄-C₁₈) alkyl (C₂-C₁₈) carboxylic ester means that either the alcohol portion or the carboxylic acid portion from which the ester is prepared bears an amino substituent NRₓR_{y}, wherein Rₓ and R_{y} are each independently a (C₁-C₈) alkyl group; in certain aspects, they are both methyl groups. Examples of such penetration enhancers include dodecyl-2-(N,N-dimethylamino) propionate (DDAIP); dodecyl-2-(N,N-dimethylamino)-acetate (DDAA); 1-(N,N-dimethylamino)-2-propyl dodecanoate (DAIPD); 1-(N,N-dimethylamino)-2-propyl myristate (DAIPM); 1-(N,N-dimethylamino)-2-propyl oleate (DAIPO); and pharmaceutically acceptable acid addition salts thereof.

In certain aspects, the skin permeation enhancer useful in the compositions of the present application is DDAIP, alone or in combination with an auxiliary permeation enhancer. DDAIP.HCl is available from Steroids, Ltd. (Chicago, Ill.) and Pisgah Laboratories (Pisgah Forest, N.C.).

The formulations described herein may include humectants, which act as hygroscopic agents, increasing the amount of water absorbed, held and retained in the pharmaceutical compositions of the application. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof.

In certain aspects of the application, the humectant is present in the composition at concentrations of from about 0.5 to about 40 percent by weight, from about 0.5 to about 20 percent by weight, and from about 0.5 to about 12 percent by weight.

The film forming agent may be a film-forming polymer comprising a vinylpyrrolidone monomer unit, including a homopolymer, (i.e., polyvinylpyrrolidone), a copolymer and a complex thereof, a gum, a resin, or the like. The term copolymer as used herein means any polymer comprising two or more different monomer repeating units and includes polymers commonly referred to as terpolymers, tetrapolymers and the like. Other film-forming agents, such as aluminum phyllosilicates (e.g. bentonite) and similar film-forming clays, may also be used.

Exemplary film-forming polymers containing vinylpyrrolidone (VP) monomer units, are polyvinylpyrrolidone (PVP), sold in a range of viscosity grades, and varying weight average molecular weights in the range of about 8,000 to about 3,000,000 Daltons (PVP K homopolymer series). PVP is sold under the trade name KOLLIDON CL by BASF Corporation. A USP grade of povidone (PVP) is one such film forming agent. Exemplary film-forming copolymers include vinylpyrrolidone/vinylaacetate (VA) copolymers available in a range of mole ratios of VP/VA such as the PVPNVA copolymer series sold by ISP, and the like. In another aspect, the polymer is a polyvinylpyrrolidone having a weight average molecular weight in the range of about 45,000 to about 60,000 Daltons.

Exemplary gums include agar gum, carrageenan gum, ghati gum, karaya gum, rhamson gum, xanthan gum and the like. Exemplary resins include carbomer, a polyacrylic acid polymer lightly cross-linked with polyalkenyl polyether. It is commercially available from Noveon Inc. (Cleveland, Ohio) under the designation CARBOPOL. An exemplary grade of carbomer is that designated as CARBOPOL 940. Other polyacrylic acid polymers suitable for use are those commercially available under the designation PEMULEN (Noveon Inc.). The PEMULEN polymers are copolymers of C₁₀ to C₃₀ alkyl acrylates and one or more monomers of acrylic acid, methacrylic acid or one of their simple esters cross-linked with an allyl ether of sucrose or an allyl ether of pentaerythritol. POLYCARBOPHIL (A. H. Robbins Company, Inc., Richmond, Va.), is another useful polyacrylic acid polymer, which is cross-linked with divinyl glycol.

The compounds described herein may be formulated with cyclodextrin or cyclodextrin derivatives, including cyclodextrin sulfobutyl ether (Capisol®, Cydex, Overland Park, Kans., USA). These and other carriers may be used to improve or otherwise modulate the solubility, penetration, uptake, and other properties of compositions comprising the compounds described herein.

The formulations described herein may assume various forms, including creams, emulsions, films, gels, lacquers, lotions, ointments, pastes, polishes (e.g. nail polish), suspensions, and the like, as well as powders, mixtures of powders and the like, emulsions, suspensions as well as solutions and gaseous formulations, such as aerosols. In other aspects, the active agent of the compositions comprising the active agent may be applied with a pipette or eye-dropper, or a cotton or fabric applicator.

There is no limitation on the form (i.e., liquid or powder) of the hydrophilic film-forming polymer used, or the amount used, as long as, the nail coat composition can be applied to the nail and forms a film thereon.

Creams are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Emulsions are two-phase systems prepared by combining two immiscible liquids, in which small globules of one liquid are dispersed uniformly throughout the other liquid. Emulsions may be designated as oil-in-water or water-in-oil type emulsions. Certain emulsions may not be classified as such because they are described by another category, such as a lotion, cream, and the like.

Gels are semisolid, suspension-type systems. Single-phase gels contain organic macromolecules distributed substantially uniformly throughout the carrier liquid, which is typically aqueous, but also, e.g., contain an alcohol such as ethanol or isopropanol and, optionally, an oil. Exemplary gelling agents include crosslinked acrylic acid polymers such as the “carbomer” family of polymers, e.g., carboxypolyalkylenes that may be obtained commercially under the Carbopol® trademark. Also useful are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions are preparations generally applied to the skin surface so as to avoid high friction, and are typically liquid or semiliquid preparations in which solid particles, including the active agent, are present in a water or alcohol base. Lotions are usually suspensions of solids, and, e.g., comprise a liquid oily emulsion of the oil-in-water type. Lotions can be used to large body areas, because of the ease of applying a generally fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Ointments are semi-solid preparations that are typically based on petrolatum or other petroleum derivatives. The specific ointment base to be used is one that will provide for optimum active ingredient delivery, and other desired characteristics, e.g., emolliency. As with other carriers or vehicles, an ointment base should be inert, stable, nonirritating and nonsensitizing. Ointment bases may be grouped in four classes: oleaginous bases, emulsifiable bases, emulsion bases and water-soluble bases. Oleaginous ointment bases include, e.g., vegetable oils, fats obtained from animals, and semisolid hydrocarbons obtained from petroleum. Emulsifiable ointment bases, also known as absorbent ointment bases, contain little or no water and include, e.g., hydroxystearin sulfate, anhydrous lanolin and hydrophilic petrolatum. Emulsion ointment bases are either water-in-oil (W/O) emulsions or oil-in-water (O/W) emulsions, and include, e.g., cetyl alcohol, glyceryl monostearate, lanolin and stearic acid. For example, water-soluble ointment bases are prepared from polyethylene glycols of varying molecular weight.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Suspensions may be defined as a coarse dispersion containing finely divided insoluble material suspended in a liquid medium.

Formulations may also be prepared with liposomes, micelles, and microspheres.

One of skill in the art, upon reading this disclosure will appreciate that any one excipient listed above can be useful in more than one manner in the formulations of the present application. That is, for example, the same excipient can be a volatile solvent and a penetration enhancer, and so on.

For example, most nail polishes are made of nitrocellulose dissolved in a solvent (e.g. butyl acetate or ethyl acetate) and either left clear or colored with various pigments. Basic components included are: film forming agents, resins and plasticizers, solvents, and coloring agents. Adhesive polymers (e.g. tosylamide-formaldehyde resin) ensure the nitrocellulose adheres to the nail's surface. Plasticizers (e.g. camphor) are chemicals that link between polymer chains, spacing them to make the film sufficiently flexible after drying. Pigments and sparkling particles (e.g. mica) add desired color and reflecting characteristics. Thickening agents (e.g. stearalkonium hectorite) are added to maintain the sparkling particles in suspension while in the bottle. Ultraviolet stabilizers (e.g. benzophenone-1) resist color changes when the dry film is exposed to direct sunlight.

In certain embodiments, the polymeric formulations may enhance the stability of the N-halogenated or N,N-dihalogenated amine compounds (some which may be unstable or degrade over time in the absence of such polymers, or in other formulations). That is, whereas certain N-halogenated or N,N-dihalogenated amine compounds referenced above may, under certain conditions, dechlorinate, react, degrade or otherwise be reduced from their original concentration or activity over time in aqueous solution, N-halogenated or N,N-dihalogenated amine compounds in certain polymeric formulations may substantially maintain their original concentration over that time frame. Such time frames may range from hours to days to months to years, depending on the chemical compound, concentration, pH, and other conditions.

The compounds described herein (including formulations thereof) may be stable, that is, they may retain a minimum concentration of N-halogenated or N,N-dihalogenated amine compound at a certain temperature or temperature range over a certain amount of time. Stability, as described herein, is generally a function of storage time and temperature. The stability of a given formulation depends generally on the particular N-halogenated or N,N-dihalogenated amine compound and other agents used in the formulation. In implementations, compounds described herein are at least 90% stable for at least 30 days at about 25° C. In certain implementations, compounds described herein are at least 90% stable for at least 90 days at about 25° C. In certain implementations, compounds described herein are at least 90% stable for at least 180 days at about 25° C.

The compounds described herein (including formulations thereof) may penetrate a nail (e.g. a human nail, e.g. a human toe nail), as penetration is describe above, within a specified time. Penetration through a nail will generally be a function of a number of factors, including various properties of the compound (e.g. molecular weight, water solubility, the partition coefficient, etc.) and the agents present in the formulation (e.g. penetration enhancers, etc.). In certain embodiments, compounds have a molecular weight between about 100 and about 300 Da, e.g., between about 100 and about 200 Da. In certain embodiments, compounds have a water solubility between about 0.1 mg/ml and about 1 g/ml in octanol-saturated water. In certain embodiments, compounds have a log P value between about −1 and about 3, e.g. between about −1 and about 2. In certain embodiments, compounds described herein penetrate a nail within about 120 hours, e.g. within about 96 hours, e.g. within about 48 hours, e.g. within about 24 hours.

Certain compounds described herein may have one or more of the properties described above. Compounds may be selected as having any combination of the properties and ranges or limits of values described above. By way of non-limiting example, certain compounds described herein may have one or more (e.g. two or more, or three or more, etc.) of the following properties: (a) stability of at least 90% for at least 30 days at 25° C.; (b) a molecular weight between about 100 and about 300 Da; (c) a water solubility between about 0.1 mg/ml and about 1 g/ml in octanol-saturated water; (d) a log P value between about −1 and about 3; and (e) penetration of a nail within about 120 hours.

In one aspect, the pharmaceutical compositions of the present applications are flowable compositions suitable for periungually or subungually forming in situ a controlled release biodegradable implant system. In another aspect, the composition comprises a biodegradable pharmaceutically acceptable thermoplastic polymer that is at least substantially insoluble in aqueous medium or body fluid, a pharmaceutically acceptable biocompatible solvent that is water soluble, and a therapeutically effective amount of an active ingredient, wherein the thermoplastic polymer and biocompatible solvent are present in concentrations effective to form the implant in situ. The active ingredient may be miscible in the polymer and/or solvent to provide a homogeneous mixture with the polymer, or insoluble to varying degrees in the polymer and/or solvent to form a suspension or dispersion with the polymer.

In one embodiment, the present application also provides implants comprising N-halogenated or an N,N-dihalogenated compounds suitable for use subungually and periungually, and methods for producing the same, as well as methods of using the implants in the treatment of diseases of the nail unit. These implants are solid articles and include microcapsules, microparticles, structured articles such as sutures, staples, medical devices, stents and the like as well as monolithic implants and implant films, filamentous membranes and matrices. In certain aspects, the implant devices are biodegradable.

In certain aspects, the pharmaceutical compositions (or simply the compositions) and implants herein are controlled-release compositions. That is, compositions that release the N-halogenated or the N,N-dihalogenated compounds and compositions over a period of time, for example, at least about 2 days, or at least about 7 days, or over a period of at least about 10 days, or at least about 14 days, or over one month or more. Certain aspects include those implants and compositions that release an active ingredient over 2 months or more, or three months or more. The composition of a suitable controlled-release composition may be tailored according to the release time required, for example the release period of the implanted composition being about 1 to about 6 months. In another aspect, the composition comprises a biodegradable polymer (poly-lactide co-glycolide) based delivery system and the polymer composition is selected to provide a release time of about 1 to about 6 months.

In certain aspects, where even longer term treatment is required, for example in onychomycosis, continuous dosing with an antifungal active may be necessary over extended periods of time, for example nine months or more. For such treatment, compositions and implants with a release period of one month, or two months, or three months, or six months, are prepared and used in the treatment repeatedly as required. Advantageously, the use of such extended release compositions can result in reduction in overall pain and discomfort to the subject during the treatment period, as well as increased patient compliance with treatment regimes. In an aspect, the composition or implant is biodegradable thus obviating the need for surgical procedures to remove the composition or implant. In another aspect, the compositions and implants are placed subdermally and sufficiently near the nail, or subungually, so as to afford accumulation of the active ingredient in and around the nail while at the same time minimizing systemic exposure.

In one aspect, the flowable compositions of the present application comprise an Atrigel biodegradable polymer (poly-lactide co-glycolide) based delivery system, wherein the polymers are dissolved in a biocompatible solvent. In other aspects, the polymers for use in the flowable compositions comprise biodegradable polymer (poly-lactide co-glycolide) based delivery systems, the blend ratio of monomers being generally about 90/10 to 10/90 (by weight) and about 25/75 through about 75/25. In other aspects, the biodegradable polymer is selected from 75/25 PLG; 85/15 PLG; 85/15 PLGH or 80/20 PLGH. Suitable biodegradable polymers for use in the compositions of the present application are those that afford release of the specific active agent over the intended period of time in situ.

Solvents suitable for use in the flowable composition are biocompatible and are at least slightly soluble in aqueous medium, body fluid, or water. The organic solvent is at least moderately soluble in aqueous medium, body fluid, or water. Testing methods to select such suitable biodegradable polymers and biocompatible solvents for use in accordance with the present application with an active ingredient are well-known to persons of skill in the art.

The flowable composition is suitable for injection under the nails of a subject where it forms a pharmaceutically acceptable, solid matrix. In one aspect of the flowable composition, a biologically active agent is included and the solid implant will release the active agent at a controlled rate. The rate of release may be altered to be faster or slower by inclusion of a rate-modifying agent that are well known in the art.

When a subject is suffering from a disease of the nail that results in separation of the nail plate from the nail bed, such as in certain forms of onychomycosis, insufficient tissue fluid may be present to afford formation of the flowable composition into a solid matrix. In such cases, the flowable composition is deposited topically in the subungual space between the nail plate and nail bed and a suitable amount of aqueous solution is introduced in a suitable manner to the composition, either simultaneously or sequentially, so as to afford formation of the solid matrix implant.

In one aspect, the present application provides injectable nanoparticulate formulations of the N-halogenated or the N,N-dihalogenated compounds that can comprise high drug (or active agent) concentrations in low injection volumes, with durations of action that can be controlled to give efficacious blood levels through manipulation of particle size and hence dissolution for periods of about one week or greater. Such composition of the application are administered via injection, such as by intramuscular or subcutaneously, to form a drug depot. The drug depot results in efficacious levels of drug up to about one week or greater. Thus, in certain aspects, the application provides compositions comprising nanoparticulate particles of N-halogenated or the N,N-dihalogenated compounds and at least one surface stabilizer.

The surface stabilizers are adsorbed to or associated with the surface of the N-halogenated or the N,N-dihalogenated compound particles. Surface stabilizers useful herein do not chemically react with the N-halogenated or the N,N-dihalogenated compound particles or itself. Individual molecules of the surface stabilizer are essentially free of intermolecular cross-linkages. The compositions can comprise two or more surface stabilizers.

Representative examples of useful surface stabilizers include but are not limited to Low viscosity hydroxypropyl cellulose (HPC or HPC-SL); hydroxypropyl methyl cellulose (HPMC); hydroxymethyl cellulose (HMC); ethylcellulose; povidone; Pluronics; sodium deoxycholate; PEG-Phospholipids; Tyloxapol and other approved tritons, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens such as e.g., Tween 20 and Tween 80 (ICI Specialty Chemicals)); polyethylene glycols (e.g., Carbowaxs 3550 and 934 (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68 and F108, which are block copolymers of ethylene oxide and propylene oxide); poloxamines (e.g., Tetronic 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic 1508 (T-1508) (BASF Wyandotte Corporation), Tritons X-200′, which is an alkyl aryl polyether sulfonate (Rohm and Haas); Crodestas F-110, which is a mixture of sucrose stearate and sucrose distearate (Croda Inc.); p-isononylphenoxypoly-(glycidol), also known as Surfactant 10-G (Olin Chemicals, Stamford, Conn.); Crodestas SL-40 (Croda, Inc.); and SA90HCO (Eastman Kodak Co.); decanoyl-N-methylglucamide; n-decyl beta-D-glucopyranoside; n-decyl beta-D-maltopyranoside; n-dodecyl beta-D-glucopyranoside; n-dodecyl beta-D-maltoside; heptanoyl-N-methylglucamide; n-heptyl-beta-D-glucopyranoside; n-heptyl beta-D-thioglucoside; n-hexyl beta-D-glucopyranoside; nonanoyl-N-methylglucamide; n-noyl beta-D-glucopyranoside; octanoyl-N-methylglucamide; n-octyl-beta-D-glucopyranoside; octyl beta-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like. Other suitable examples are provided in US Pat. App. Pub. No. 20060154918.

The injectable nanoparticulate formulations (for depot injection) of the N-halogenated or an N,N-dihalogenated compounds of the application can be prepared following methods described in U.S. Pat. App. Pub. No. 2006/0154918, upon appropriate substitution of the active ingredient. In one aspect, the method comprises of one of the following methods: attrition, precipitation, evaporation, or combinations of these. Exemplary methods of making nanoparticulate compositions are also described in U.S. Pat. No. 5,145,684.

In other aspects of the application, the injectable nanoparticulate formulations of the present application are injected subungually or periungually for the treatment of onychomycosis. In certain other aspects, the injectable nanoparticulate formulations of the present application are injected under the skin which is near the nail.

The current disclosure also relates to methods for treating or preventing infections of the nail, claw, or hoof, comprising administering to a subject in need thereof a composition or formulation comprising (i) a first active agent comprising an N-halogenated or N,N-dihalogenated amine compound of Formula (I); and (ii) a second active agent, wherein the second active agent is an anti-infective agent of a different class than the first active agent.

Such second active agents include, but are not limited to oral antifungal agents such as terbinafine, itraconazole, and fluconazole, as well as topical antifungal agents such as allylamines (e.g. terbinafine), triazoles (e.g. itraconazole and fluconazole), imidazole derivatives (e.g. ketoconazole, miconazole, clotrimazole, and econazole), amorolfine, ciclopirox, alamine, sodium pyrithione, bifonazole plus urea, and propylene glycol plus urea plus lactic acid. Oral and topical antibacterial, antiviral, and other anti-infective agents may also be used. Such agents include, but are not limited to, benzyl alcohol, phenoxyethanol, phenethylalcohol, iodopropynyl butyl carbamate, paraben, quaternary ammonium compounds (e.g., benzalkonium chloride), benzoyl peroxide, and chlorhexidine.

The current disclosure also relates to a device or apparatus for treating or preventing infections of the nail, claw, or hoof, wherein the device or apparatus enables application of an N-halogenated or N,N-dihalogenated amine compound of Formula (I) to the nail or nearby tissue of a subject in need thereof. Suitable devices such as bandages and patches are described in more detail below.

### EXAMPLES

### Example 1

### Application of Formulations on Infected Human Nail Samples

Preparation of a Suspension of Trichophyton rubrum (T. rubrum)

A laboratory-cultured clinical isolate of T. rubrum was used to seed A 90 mm SDA plate. Mycelium and spores, using a sterile swab, were removed from a slope culture and transferred onto the surface of the agar. The agar plate was then incubated at 25° C. for 7 days. The white spores were then washed from the surface of the plate with Ringers solution. The spore suspension was filtered through sterile gauze to remove mycelium and agar debris. A viable count of the spore suspension was performed and the spore count adjusted to approximately 1×107 cfu/ml, by diluting or concentrating the spores accordingly in a final volume of 20 ml.

### Nail Preparation

Grown, clipped human nails were used for this experiment. Prior to cutting the nails into 3 mm×3 mm segments, the nails were removed from the freezer and placed in a laminar flow cabinet for 30 min to equilibrate at room temperature. Following this the nails were briefly washed separately by ethanol and water separately. The nails then were washed with sterile Ringer's solution and placed into a sterile Petri dish without a lid and air dried under a laminar flow cabinet for 30 min at room temperature.

### Nail Test System

Quenching: The quenching effect of all the formulations was tested (n=6) by adding 2 μL of each formulation into a known concentration of ATP calibration standard (50.0 ng/mL) and measuring the amount of ATP. A control was also set up with the standard only. The data generated was processed as a percentage of ATP recovered in the presence of various matrices compared to the standard on its own. This data was used to evaluate several statistical possibilities for the final calculation of efficacy.

### Preparation of ChubTur® Cells

A ChubTur® cells test system (MedPharm Ltd., Guildford, UK) was assembled. The ChubTur® cells were used as follows:

Full thickness toe nails were disinfected by washing in 70% ethanol solution, followed by rinsing, cut into 3 mm×3 mm segments, measured for thickness and infected using T. rubrum cell suspension (5 μL of ˜1×107 cfu/ml).

At 14 days, the ChubTur® cells were removed from incubation at 25° C. and 2 μL of the Test items applied to the surface of the nail, opposite to where the nail was inoculated with the organism suspension.

The nail samples were dosed using the respective test compositions for 7 days.

After incubation, the ChubTur® cells with the test compositions applied were removed from incubation. The excess test compositions were removed from the surface of the nails and the nails were dismantled from the ChubTur® cells.

All the nails were then analyzed for the presence of ATP from the viable fungi.

### Results

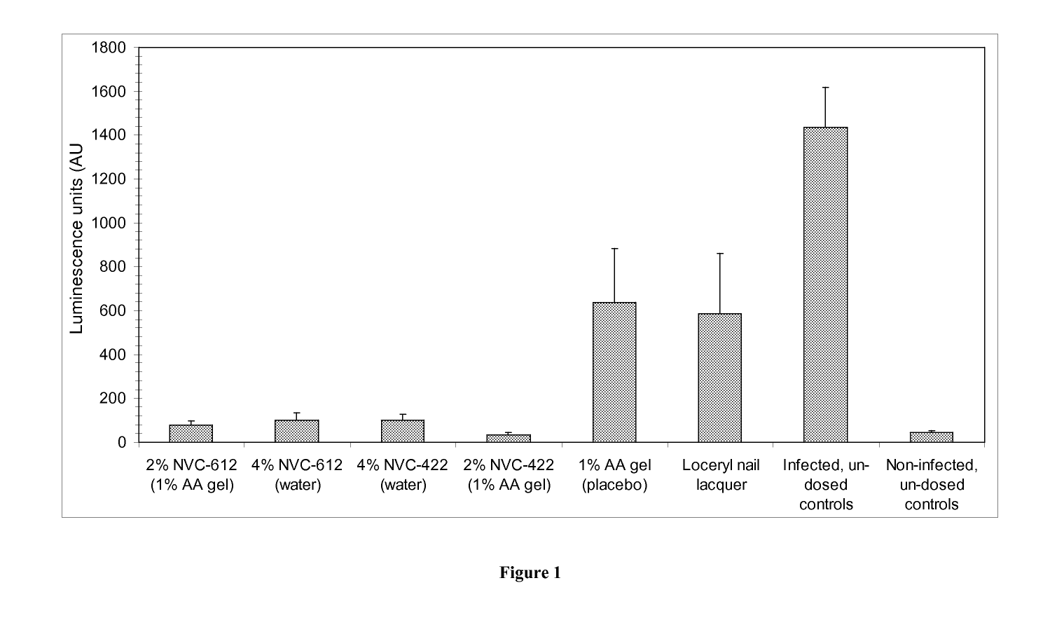

FIG. 1 compares ATP release (mean±SD) following application of test compositions over 7 day incubation periods (n=6) and undosed controls (n=3). The test compositions used in this example were 2% N-chloro-2,2-dimethyltaurine (“NVC-612”) in 1% AA1 gel (“AA gel”); 4% NVC-612 in water; 2% N,N-dichloro-2,2-dimethyltaurine (“NVC-422”) in 1% AA gel; 2% NVC-422 in water; 1% AA gel as a negative control or placebo; and Loceryl® nail lacquer as a positive control.

The results of the infected nail study following a single 2 μl dosage of each test composition to the first (e.g. dorsal) surface of human toe nails infected on the second (e.g. ventral) surface with T. rubrum show that all N-halogenated and N,N-dihalogenated compounds tested were statistically more effective at reducing ATP production than both the marketed comparator Loceryl® and the placebo formulation (1% AA gel), and that the formulation of 2% NVC-422 in 1% AA gel was the most effective.

### Example 2

### Topical Gel Formulation

A topical gel formulation of the N-halogenated and N,N-dihalogenated compounds of the present application is prepared by conventional pharmaceutical methods. In one example, the indicated amounts of the following ingredients are used in a formulation:

| Ingredient | Amount |
|---|---|
| N,N-dichloro-2,2-dimethyltaurine | 30 grams |
| Purified water | 600 grams |
| Polyethylene glycol | 400 grams |
| Potassium hydroxide | 0.01 gram |
| Edetate disodium | 0.1 gram |
| Carbomer 934P | 12.5 grams |
| Poloxamer 407 | 2.0 grams |
| Polysorbate 40 | 2.0 grams |
| Butylated hydroxytoluene | 0.5 grams |
| Benzyl alcohol | 10.0 grams |

The N,N-dichloro-2,2-dimethyltaurine, the carbomer 934P and the edetate disodium are added to 250 mL of the purified water, and the mixture is homogenized at low speed until the carbomer is dispersed. Next, the poloxamer 407, mixed with 250 mL of the purified water, is added to the carbomer mixture, and the resulting mixture is homogenized at low speed. The potassium hydroxide, dissolved in 100 mL of purified water, is added to this mixture, and the resulting mixture (Mixture 1) is homogenized at low speed. In a separate container, the polysorbate 40 and the butylated hydroxytoluene are added to the polyethylene glycol, and the resulting mixture is heated to 65° C. and maintained at this temperature until all the compounds are dissolved. The mixture is then allowed to cool to room temperature, at which time the benzyl alcohol is added, and the resulting mixture is homogenized at low speed. This mixture is then added to Mixture 1, and the resulting mixture is mixed at low speed until it is homogeneous, forming a gel of the application.

### Examples 3-5

### Additional Topical Gel Formulations

Topical gel formulation of other compounds described herein may be prepared as in Example 2, substituting the following compounds for N,N-dichloro-2,2-dimethyltaurine:

Example 3: 3-(chloroamino)-N,N,N-3-tetramethylbutan-1-ammonium chloride.

Example 4: 1-(3-(dichloroamino)-3-methylbutyl)-4,4-difluoro-1-methylpiperidinium chloride;

Example 5: (3-chloro-4-methyl-2-oxooxazolidin-4-yl)methanesulfonic acid;

Other compounds described herein may also be used in the gel, cream, patch, and other formulations described herein.

### Example 6

### Topical Cream Formulation

A topical cream formulation of the N-halogenated and N,N-dihalogenated compounds of the present application is prepared by conventional pharmaceutical methods. In one example, the indicated amounts of the following ingredients are used in a formulation:

| Ingredient | Amount |
|---|---|
| N,N-dichloro-2,2-dimethyltaurine | 30 grams |
| Purified water | 370 grams |
| White petrolatum | 250 grams |
| Stearyl alcohol | 250 grams |
| Propylene glycol | 120 grams |
| Sodium lauryl sulfate | 10 grams |
| Methylparaben | 0.25 gram |
| Propylparaben | 0.15 gram |
| Potassium hydroxide | 0.01 gram |

The stearyl alcohol and the white petrolatum are melted together on a steam bath, and then maintained approximately at room temperature (in certain instances, depending on the nature of the anti fungal compound of the application employed in the topical cream, the mixture can be maintained at a higher temperature of, for example, approximately 75° C.). The other ingredients are then added, after previously having been dissolved in the purified water (the aqueous solutions may be warmed to, say, 75° C., prior to mixing, depending on the nature of the anti fungal compound of the application employed in the topical cream), and the resulting mixture is stirred until it congeals into a cream.

### Example 7

### Nail Patch

A nail patch of the composition of the present application is prepared by conventional pharmaceutical methods. A square piece of sterile, finely woven gauze one centimeter on each side is placed in the center of a square piece of occlusive surgical adhesive tape two centimeters on each side. To the gauze is applied 0.4 mL of the gel of Example 2; the gel is allowed to soak into the gauze. This nail patch is prepared and can be stored for up to about 2 years before being applied to a subject to treat or prevent an infection of a nail, claw, or hoof.

Additional examples of patches are provided below.

### Example 8

### Treatment of Onychomycosis with Gel Formulation

The gel formulation of Example 2 is provided to human subjects having distal subungual onychomycosis. Each infected nail to be treated is examined, cultured, and photographed before treatment begins. Culturing is performed as described by B. Elewski (Journal of the American Academy of Dermatology, v. 35 (number 3, part 2): S6-S9, incorporated herein by reference): The nail to be sampled is first swabbed liberally with alcohol to eliminate as many bacteria as possible, as bacteria could overgrow and inhibit the growth of fungi. A small curette or special nail clipper is used to cut away the distal end of the nail plate; a curette is then used to scrape debris from the nail bed at a site as close to the cuticle as possible; scrapings from the under surface of the nail plate may be included. The shavings specimen is divided in half, each half being spread on a petri dish containing Sabouraud glucose agar. One petri dish is cycloheximide-free, while the other contains cycloheximide in the agar. Chloramphenicol is usually added to the culture media in both dishes to inhibit bacterial growth. The dishes are incubated for 7 to 14 days and then examined microscopically to identify any fungal or yeast growth. As cycloheximide inhibits nondermatophyte growth but not dermatophyte growth, colonies that appear on both dishes are likely to be dermatophytes, while those that only appear on the cycloheximide-free dish are likely to be nondermatophyte species.

Each of the subjects is instructed to topically apply the formulation to the affected nail or nails twice daily for eight weeks. The subjects return to the clinic every seven days, when each nail is again examined, cultured and photographed.

### Example 9

### Treatment of Onychomycosis with Patches

The patches of Example 7 are provided to subjects having onychomycosis on one or more nails, such as fingernails. Each infected nail to be treated is examined, cultured, and photographed before treatment begins. Culturing is performed as in Example 4. Each of the subjects is instructed to topically apply such a skin patch to the affected nail or nails, replacing the old patch with a new one every 48 hours. The subjects return to the clinic every seven days, when each nail is again examined, cultured and photographed.

### Example 10

### Treatment of Onychomycosis with Cream Formulation

The cream formulation of Example 6 is provided to subjects having white superficial onychomycosis. Each infected nail to be treated is examined, cultured, and photographed before treatment begins. Culturing is performed as in Example 5, except that the specimen is obtained by scraping debris from the surface of the infected nail with a No. 15 blade scalpel or a sharp curette. Each of the subjects is instructed to topically apply the formulation of the present application to the affected nail or nails twice daily for eight weeks. The subjects return to the clinic every seven days, when each nail is again examined, cultured and photographed.

### Example 11

### Patches

This example provides further detail of patches of the present application.

### Patches and Methods

As one of skill in the art will appreciate upon reading this disclosure, the single-layer matrix patch (or devise) comprises a backing medium, a single adhesive matrix layer and a detachable outer covering. The multi-layer matrix device comprises at least two matrix layers, at least one of the two layers being an adhesive layer. Each is separated by a membrane which allows gradual release of the compounds of the present application, optionally in combination with other active agents.

Backing Medium Layers

The backing medium of the patch is the part of the patch that is visible when it is applied on the skin. It serves to maintain the active agent in contact with the nail to increase the diffusion of the active agent inside the nail. It may be occlusive or non-occlusive. The backing medium also serves to protect the layer in contact with the nail against any contamination. The backing media used in these examples are Dow BLF 2023 and Dow BLF 2080.

### Adhesives

For the purposes of this example, the term “adhesive” denotes a polymer or a mixture of polymers that are chemically inert as to the different components of the matrix, including in particular the compounds of the present application and optionally, absorption promoters. Its principal function is to ensure the adhesion of the patch on the nail. Patch materials may be selected so that the patch leaves little or no residue on the nail when the patch is removed, and so that it exhibits good adherence with respect to the backing medium.

An exemplary patch material is any acrylic-based material. For example, an acrylic-based adhesive can have a base of (meth)acrylic acid ester copolymer containing as a monomer component a (meth)acrylic acid alkyl ester in which the alkyl has about 4 to about 12 carbon atoms. Examples of monomers include n-butyl acrylate, n-hexyl acrylate, isooctyl acrylate, and so forth, as provided in, e.g., U.S. patent application Ser. No. 12/226,375 (Kawahara et al.).

The acrylic polymer used in this example is a polymer in solution in ethyl acetate marketed principally under the trademark Duro-Tak 387-2516 by National Starch. Alternatively, the silicone polymer BIO PSA 7-4102, a polymer in solution in ethyl acetate supplied by Dow Corning, is also useful.

Matrix or Adhesive Matrix Layer

The term matrix or adhesive matrix layer means a mixture of at least one active agent including a compound of the application, such as, e.g., N,N-dichloro-2,2-dimethyltaurine sodium salt, homogeneously dispersed or dissolved in a biocompatible and pressure-sensitive adhesive, wherein the mixture optionally containing promoters to enhance the absorption of the homogeneously dissolved or dispersed compounds of the application. In the case of a single-layer device, this matrix layer has a surface directly in contact with the backing medium, and a second surface directly in contact with the detachable outer covering to be removed when the patch is applied on the area to be treated. During use, this detachable outer covering is removed from the surface of the matrix layer which is then applied directly onto the nail. The active agent and the absorption promoters diffuse through the adhesive and its mixtures, if any, to the surface of the nail.

Detachable Outer Covering

The detachable outer covering 3M 9956 used in this example is a polyester film supplied by 3M. The side in contact with the substrate is treated with a fluorinated polymer. It is compatible with the adhesives used. It is transparent, heat sensitive and its thickness is 74+/−5 μm.

### General Procedure for the Preparation of Patches

The patches are prepared by the solvent phase coating technique including: mixing of all the constituents of the adhesive matrix layer in the solvent, the adhesive being necessarily miscible in this solvent to ensure good coating homogeneity; the mixture is stirred using a magnetic stirrer until a homogeneous preparation is obtained; about 30 g of the preparation are taken to apply the coating of the more or less viscous solution obtained onto the backing medium; finally, evaporating the solvent, in the air under an extraction hood for 24 hours at ambient temperature. The coating is performed using an Erichsen KCC 101 film applicator with 400 μm or 100 μm thick spiral, wire coating bars.

### Analysis of the Patches

The possible formation of sodium salt crystals of the active agent and the coating uniformity of the patches are observed under a microscope at a magnification ranging from ×50 to ×250 and the images are taken using the “Kappa” image acquisition software.

### Microbiological Evaluation Procedure

This test serves to determine the efficacy of these patches of the application. The experiment is conducted on pig's hooves which are physiologically similar to human nails. The patches are placed on pig's hooves that have first been rehydrated. The hooves are placed on a Sabouraud agar (AES Laboratory reference AEB 152352) infected by an inoculum of spores of T. rubrum, which is implicated in onychomycoses. The agar samples and hooves are then placed in a refrigerator at +4° C. for a determinate period to allow the active agent to diffuse through the hoof then the agar. The agar samples are then placed in an incubator for 48 hours at 32.5° C., which is the ideal temperature for the proliferation of T. rubrum.

The experiment is performed, in sterile conditions, in several steps: rehydration of the pig's hooves, preparation of the infected agars, application of patches on the pig's hooves, refrigeration at 4° C. for a defined period, incubation at 32.5° C. for 48 hours.

Rehydration of Pig's Hooves

The pig's hooves are first scraped to remove all of the flesh and are then autoclaved. It is then necessary to rehydrate them before use. A zone delineated by a ring indicates an area on which the active agent is deposited. This ring should be manually glued to the hoof before rehydration. These two steps are performed at the same time: place a sterile gauze in a round Petri dish, moisten with sterilized water, cut out the roundels, place glue on the roundels, take a hoof, check its integrity, glue the roundel onto the hoof, making sure that no excess glue appears inside the roundel, place the hoof on the moist gauze and leave while preparing the culture.

Preparation of Infected Agars

Take up the working cultures in inclined tubes, scrape these cultures with Sabouraud broth (AES Laboratory reference 111 105) to collect the spores, filter the suspension to eliminate all residues of agar, and dilute so as to have sufficient suspension to infect the agars (i.e., 1 ml of suspension per 100 ml of agar). Make a 10-fold dilution of the suspension, place the dilution in a Thoma cell, leave to rest for a quarter of an hour, and count the spores under the microscope. Calculate the number of colonies in the suspension, raise this number to the power 7 or 8, if the concentration is good the procedure continues (if not, dilute the initial solution or re-scrape the cultures to collect new spores).

Place the solution in a vortex stirrer to ensure good homogeneity, take the necessary quantity of suspension, add the suspension to the Sabouraud agar, and stir well. Keep the dishes flat using a level and modeling paste, close to a Bunsen burner, fill the Petri dishes by pouring 250 ml of infected agar into each dish, leave to set en masse, leaving the dish lids ajar. Close the dishes, place them under a laminar flow hood, fully open the dishes; leave them to dry completely for half an hour.

Application of Patches

Cut out a sample using a die-cutter of precise diameter (8 mm), paste it onto the hoof inside the roundel, place the hooves on the agar, with four hooves per dish, then place the dishes in the refrigerator (+4° C.) for a determinate period.

### Diffusion and Incubation

Diffusion of the active agent through the hoof and the agar takes place during the refrigeration period as the trychophyton is unable to grow. Incubation corresponds to the period during which the fungus is able to grow. The final steps are as follows. Remove the dishes from the refrigerator after the specified time, remove the hooves using tweezers, place the dishes in an oven (+32.5° C.) to allow the T. rubrum to develop; 48 hours is generally a sufficient period in which to obtain clear inhibition zones.

### Results

When the fungus has grown sufficiently, inhibition zones appear indicating the diffusion of active agent through the hoof to the agar. The inhibition zone is larger or smaller depending on the degree of diffusion. The results are determined by comparing these zones. The operating protocol is as follows. Count the inhibition zones, cut out and identify these zones, weigh each zone. Evaluate each formulation on a points scale (n=3).

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating or preventing an infection of a nail, claw, or hoof comprising administering to a subject a pharmaceutical composition comprising an N-halogenated or N,N-dihalogenated amine compound.

2. The method of claim 1 wherein the compound is a compound of Formula (I)
A-C(R¹R²)R(CH₂)ₙC(R³R⁴)—Y—Z (I)
or a derivative thereof, wherein
A is hydrogen, HalNH— or Hal₂N—, wherein Hal is a halogen selected from the group consisting of chloro, bromo and iodo;
R¹ is hydrogen or an optionally substituted group selected from the group consisting of alkyl, cycloalkyl, heteroalkyl, aryl, heteroaryl, and heterocycloalkyl groups, and —COOH;
R² is hydrogen or an optionally substituted group selected from the group consisting of alkyl, cycloalkyl, heteroalkyl, aryl, heteroaryl, and heterocycloalkyl groups, or R¹ and R² together with the carbon atom to which they attach form an optionally substituted cycloalkyl or heterocycloalkyl group;
R is a carbon-carbon single bond or a divalent cycloalkylene radical with three to six carbon atoms,
n is 0 or an integer from 1 to 13;
R³ and R⁴ are each independently selected from the group consisting of hydrogen, fluoro, —NHHal, NHal₂, and an optionally substituted group selected from the group consisting of alkyl, cycloalkyl, heteroalkyl, aryl, heteroaryl, and heterocycloalkyl groups;
Y is selected from a group consisting of a single bond; —O—, —CF₂—, —CHF—, —C(═O)—, —C(═O)O—, —OC(═O)—, —C(═O)NR^{a}—, —NR^{a}C(═O)—, —P(═O)(OR^{b})O—, —OP(═O)(OR^{b})—, —P(═O)(OR^{b})NR^{c}—, —NR^{c}P(═O)(OR^{b})—, —S(═O)₂, —S(═O)₂O—, —OS(═O)₂—, —S(═O)₂NR^{d}—, —NR^{d}S(═O)₂—, or heteroaryl wherein R^{a}, R^{b}, R^{c} and R^{d} are each independently selected from the group consisting of hydrogen, and optionally substituted alkyl, aryl, cycloalkyl, heteroalkyl, heteroaryl, and heterocycloalkyl; a divalent (C₁₋₁₈)alkyl group in which, optionally, one or two methylene groups are replaced with a mono- or di-substituted methylene group; and a divalent (C₁₋₁₈)heteroalkyl group wherein the divalent (C₁₋₁₈)heteroalkyl group is a divalent (C₁₋₁₈)alkyl group in which, optionally, one or two methylene groups are replaced with 1 or 2-NR′—, —O—, —S—, —S(═O)—, >C═O, —C(═O)O—, —OC(═O)—, —C(═O)NH—, —NHC(═O)—, —C(═O)NR′—, —NR′C(═O)—, —S(═O)₂—, —S(═O)₂NR′—, —S(═O)₂NH—, —NR′S(═O)₂— or —NHS(═O)₂—, wherein R′ is selected from the group consisting of hydrogen, Cl, Br, and optionally substituted alkyl, aryl, cycloalkyl, heteroalkyl, heteroaryl, heterocycloalkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, and (C₆₋₁₀)aryl(C₁₋₄)alkylC(═O)— wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄) alkyl, the heterocycloalkyl group containing 2-10 carbon atoms and 1 to 4 heteroatoms selected from N, O or S;
Z is selected from the group consisting of hydrogen, —CO₂H, —CONH₂, —SO₃H, —SO₂NH₂, —P(═O)(OH)₂, —B(OH)₂, —[X(R⁵)(R⁶)R⁷]Q, —S(═O)₂NR^{c}R^{d}, —S(═O)₂NHC(═O)R^{e}, S(═O)₂C(═O)NR^{c}R^{d}, —S(═O)₂NR^{c}C(═O)NR^{c}R^{d} and —S(═O)₂(N═)C(OH)NR^{c}R^{d} wherein R^{c} and R^{d} are each independently hydrogen or is independently selected from the group consisting of (C₁₋₅)alkyl, (C₃₋₆)cycloalkyl, (C₁₋₅)alkylNHC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl(C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and R^{e} is hydrogen or is selected from the group consisting of (C₁₋₅)alkyl, (C₃₋₆)cycloalkyl, (C₆₋₁₄)aryl, (C₆₋₁₀)aryl(C₁₋₄)alkyl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S;
X is selected from the group consisting of N, P, and S;
Q is a counter anion or is absent;
R⁵ and R⁶ are each independently selected from the group consisting of alkyl, aryl, cycloalkyl, heteroalkyl, heteroaryl, and heterocycloalkyl, each of which may be optionally substituted; or R⁵ and R⁶ together with the X atom to which they are attached form heterocycloalkyl group, which may be optionally substituted; and
R⁷ is alkyl, aryl, cycloalkyl, heteroalkyl, heteroaryl, or heterocycloalkyl, each of which may be optionally substituted, and may further be O when X is N, with the proviso that R⁷ is absent when X is S;
and with the proviso that if R is a divalent cycloalkylene radical, n will not exceed the integer 11.

3. The method of claim 2 wherein the compound is a compound of Formula (IA)
A-C(R¹R²)R(CH₂)ₙ—C(R³R⁴)—Y—Z (IA)
or a derivative thereof, wherein
A is hydrogen, Hal₂N—, or HalHN, wherein Hal is halogen selected from the group consisting of chloro, bromo and iodo;
R¹ is hydrogen, (C₁₋₆)alkyl or the group —COOH;
R² is hydrogen or (C₁₋₆)alkyl, or R¹ and R² together with the carbon atom to which they attach form a (C₃₋₆)cycloalkyl ring;
R is a carbon-carbon single bond or a divalent cycloalkylene radical with three to six carbon atoms;
n is 0 or an integer from 1 to 13,
R³ is hydrogen, (C₁₋₆)alkyl, —NHHal, or —NHal₂;
R⁴ is hydrogen or (C₁₋₆)alkyl;
Y is a single bond;
and Z is selected from the group consisting of hydrogen, —CO₂H, —CONH₂, —SO₃H, —SO₂NH₂, —P(═O)(OH)₂ and —B(OH)₂.

4. The method of claim 2 wherein the compound is a compound of Formula (IB)
A-C(R¹R²)—C(R³R⁴)—Y—Z (IB)
or derivative thereof, wherein
A is selected from the group consisting of hydrogen, Hal₂N—, and HalHN;
Hal is halogen selected from the group consisting of chloro and bromo;
R¹ and R² are each independently selected from the group consisting of (C₁₋₅)alkyl, heteroalkyl, halo(C₁₋₅)alkyl, (C₃₋₆)cycloalkyl, (C₃₋₆)cycloalkyl(C₁₋₃)alkyl, (C₆₋₁₀)aryl(C₁₋₄)alkyl, (C₆₋₁₄)_{aryl}, heteroaryl, and (C₃₋₁₀)heterocycloalkyl, or R₁ and R₂ together with the carbon atom to which they are attached to form a (C₃₋₁₂)cycloalkyl or (C₃₋₁₂)heterocycloalkyl;
R³ and R⁴ are each independently selected from the group consisting of hydrogen, fluoro, (C₁₋₅)alkyl, heteroalkyl, halo(C₁₋₅)alkyl, (C₃₋₆)cycloalkyl, (C₃₋₆)cycloalkyl(C₁₋₃)alkyl, (C₆₋₁₀)aryl(C₁₋₄)alkyl, (C₆₋₁₄)aryl, heteroaryl, and (C₃₋₁₀) heterocycloalkyl, or R³ and R⁴ together with the carbon atom to which they are attached to form a (C₃₋₁₂)cycloalkyl, or (C₃₋₁₂) heterocycloalkyl;
Y is selected from a group consisting of single bond, —O—, a divalent (C₁₋₁₈)alkyl group in which optionally one or two methylene groups are replaced with a mono- or di-substituted methylene group, and a (C₁₋₁₈)heteroalkyl group,
with the proviso that when R¹ is (C₁₋₅)alkyl or when R¹ and R² together with the carbon atom to which they attach form a (C₃₋₆)cycloalkyl, then Y must be —O— or a divalent (C₁₋₁₈) alkyl group wherein one or two methylene groups are replaced with a substituted methylene group or Y must be a divalent (C₁₋₁₈) heteroalkyl group wherein the (C₁₋₁₈) heteroalkyl group is a (C₁₋₁₈)alkyl group where one or two methylene groups are replaced with a by —NR′—, —O—, —S—, —S(═O)— or —S(═O)₂—;
R′ is hydrogen or is selected from the group consisting of Cl, Br, (C₁₋₅)alkyl, (C₃₋₆)cycloalkyl, (C₆₋₁₀)aryl(C₁₋₄)alkyl, (C₁₋₅)alkylNHC(═O)—, (C₁₋₅)alkoxyC(═O)—, R^{a}R^{b}NC(═O)—, (C₁₋₅)alkylC(═O)—, (C₆₋₁₀)arylC(═O)—, (C₆₋₁₀)aryl(C₁₋₄)alkylC(═O)—, (C₆₋₁₄)aryl, heteroaryl comprising 4 to 10 ring atoms with at least one heteroatom selected from O, S and N in the ring, and heterocycloalkyl containing 2-10 carbon atoms and 1 to 4 heteroatoms selected from N, O or S, and wherein R^{a} and R^{b} are each independently hydrogen, (C₁₋₅)alkyl, (C₃₋₆)cycloalkyl, (C₁₋₅)alkylNHC(═O)—, (C₁₋₅)alkylC(═O)—, (C₆₋₁₄)aryl, (C₆₋₁₀)aryl(C₁₋₄)alkyl, heteroaryl comprising 4 to 10 ring atoms with at least one heteroatom selected from O, S and N in the ring, or heterocycloalkyl(C₁₋₄) alkyl, the heterocycloalkyl group containing 2-10 carbon atoms and 1 to 4 heteroatoms selected from N, O or S; and
Z is selected from the group consisting of hydrogen, —CO₂H, —CONH₂, —SO₃H, —SO₂NH₂, —P(═O)(OH)₂ and —B(OH)₂.

5. The method of claim 2 wherein the compound is a compound of Formula (IC)
A-C(R¹R²)(CH₂)ₙY(CH₂)ₘ—Z (IC)
or a derivative thereof, wherein:
A is HalHN— or Hal₂N—, wherein Hal is halogen selected from the group consisting of chloro and bromo;
R¹ and R² are each independently selected from the group consisting of alkyl, aryl, cycloalkyl, heteroalkyl, heteroaryl, and heterocycloalkyl, each of which may be optionally substituted; or R¹ and R² together with the carbon atom to which they are attached form a cycloalkyl or heterocycloalkyl group, each of which may be optionally substituted;
Y is selected from the group consisting of a single bond, —O—, —CF₂—, —CHF—, —C(═O)—, —C(═O)O—, —OC(═O)—, —C(═O)NR^{a}—, —NR^{a}C(═O)—, —P(═O)(OR^{b})O—, —OP(═O)(OR^{b})—, —P(═O)(OR^{b})NR^{c}—, —NR^{c}P(═O)(OR^{b})—, —S(═O)₂, —S(═O)₂O—, —OS(═O)₂—, —S(═O)₂NR^{d}—, —NR^{d}S(═O)₂—, or heteroaryl, wherein R^{a}, R^{b}, R^{c} and R^{d} are each independently selected from the group consisting of hydrogen, and optionally substituted alkyl, aryl, cycloalkyl, heteroalkyl, heteroaryl, and heterocycloalkyl;
Z is —[X(R⁵)(R⁶)R⁷]Q, wherein Q, R⁵, R⁶, and R⁷ are defined as in Formula (I) above;
n is 0 or is an integer from 1 to 12; and
m is an integer from 1 to 12.

6. The method of claim 2 wherein the compound is a compound of Formula (ID)
A-C(R₁R₂)(CH₂)ₙC(R³R⁴)—Z (ID)
or a derivative thereof, wherein:
A is hydrogen, HalNH— or Hal₂N—, wherein Hal is halogen selected from the group consisting of chloro, bromo and iodo;
R¹ and R² are each independently selected from an optionally substituted group selected from the group consisting of alkyl, cycloalkyl, heteroalkyl, heteroaryl, and heterocycloalkyl groups, or R¹ and R² together with the carbon atom to which they attach form a (C₃₋₆)cycloalkyl ring;
n is 0 or an integer from 1 to 13
R³ and R⁴ are independently selected from the group consisting of hydrogen, fluoro, and an optionally substituted group selected from the group consisting of alkyl, cycloalkyl, heteroalkyl, heteroaryl, and heterocycloalkyl groups;
Z is selected from the group consisting of, —SO₃H, —SO₂NH₂, —P(═O)(OH)₂, —B(OH)₂, and —[X(R⁵)(R⁶)R⁷]Q, R⁵, R⁶, and R⁷ is defined as in Formula (I) above; and
n is 0 or an integer from 1 to 6.

7. The method of claim 1 wherein the compound is a compound of Formula (II) 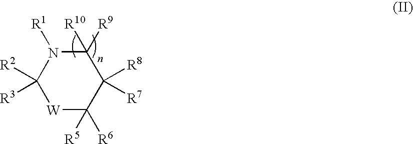
or a derivative thereof, wherein:
n is 0 or 1;
W is NR⁴, O, S, S(═O) or S(═O)₂;
R¹ is H, Cl, Br, -L-X or optionally substituted alkyl or heteroalkyl;
R² and R³ are each independently H, -L-X, or optionally substituted alkyl or heteroalkyl, or R² and R³ together with the carbon to which they are attached form a carbonyl, -L-X or an optionally substituted cycloalkyl or heterocycloalkyl group;
R⁴ is H, Cl, Br, -L-X or optionally substituted alkyl or heteroalkyl;
R⁵ and R⁶ are each independently H, -L-X or optionally substituted alkyl or heteroalkyl; or R⁵ and R⁶ together with the carbon to which they are attached form a carbonyl, -L-X or an optionally substituted cycloalkyl or heterocycloalkyl group;
R⁷ and R⁸ are each independently H, -L-X or optionally substituted alkyl or heteroalkyl; or R⁷ and R⁸ together with the carbon to which they are attached form a carbonyl, -L-X or an optionally substituted cycloalkyl or heterocycloalkyl group;
R⁹ and R¹⁰ are each independently H, -L-X or optionally substituted alkyl or heteroalkyl; or R⁹ and R¹⁰ together with the carbon to which they are attached form a carbonyl, -L-X or an optionally substituted cycloalkyl or heterocycloalkyl group;
each L is independently an optionally substituted C₁₋₆ alkyl, heteroalkyl, cycloalkyl or heterocycloalkyl group; and
each X is independently —SO₃H, —N⁺R^{a}R^{b}R^{c}, —B(OH)₂, —CO₂H, —PO₃H₂ or —PO₃HR^{a} and R^{a}, R^{b}, and/or R^{c} are independently a bond or an optionally substituted alkyl or heteroalkyl groups, or may form, together with the N to which they are attached, a heterocycloalkyl group;
with the provisos that:
at least one of R¹, R², R³, R⁴, R⁵, R⁶, R⁷, R⁸, R⁹ or R¹⁰ is -L-X; and
at least one of R² and R³, R⁵ and R⁶, or R⁷ and R⁸, together with the carbon to which they are attached, form a carbonyl; provided that (i) R⁵, R⁶ and the carbon to which they are attached, and R⁷, R⁸ and the carbon to which they are attached, are not both carbonyl; and (ii) R⁷, R⁸ and the carbon to which they are attached, and R⁹, R¹⁰ and the carbon to which they are attached, are not both carbonyl.

8. The method of claim 1 wherein the compound is a compound of Formula (III) 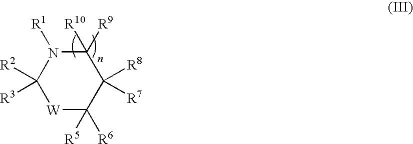
or a derivative thereof, wherein
n is 0 or 1;
W is NR⁴ or O;
R¹ is H, Cl, Br, or optionally substituted alkyl;
R², R³, R⁵, R⁶, R⁷, R⁸, R⁹, and R¹⁰ are each independently H or optionally substituted alkoxy, alkyl, heteroalkyl, aryl, heteroaryl, arylalkyl, or hydroxyl; or R² and R³ together with the carbon to which they are attached, R⁵ and R⁶ together with the carbon to which they are attached, R⁷ and R⁸ together with the carbon to which they are attached, and/or R⁹ and R¹⁰ together with the carbon to which they are attached form a carbonyl or an optionally substituted cycloalkyl or heterocycloalkyl group;
R⁴ is H, Cl, Br, or optionally substituted alkyl, with the proviso that R¹ and R⁴ are not both H.

9. The method of claim 1 wherein the compound is selected from the group consisting of
N,N-dichlorotaurine;
N,N-dichloro-2-methyltaurine;
N,N-dichloro-2,2,3,3-tetramethyl-β-alanine;
N,N-dichloro-2,2-dimethyltaurine;
N,N-dichloro-1,1,2,2-tetramethyltaurine;
N,N-dibromo-2,2-dimethyltaurine;
N,N-dibromo-1,1,2,2-tetramethyltaurine;
N,N-diiodotaurine;
N,N-dichloro-3,3-dimethylhomotaurine;
N,N-dichloro-2-methyl-2-amino-ethanesulfonic acid; and
N,N-dichloro-1-methyl-ethanesulfonic acid,
N,N-dichloro amino-trimethylene phosphonic acid;
N,N-dibromo-2-amino-5-phosphonopantanoic acid;
N,N-dichloro amino-ethylphosphonic acid diesters;
N-chloro amino-ethylphosphonic acid diethylester;
N-chloro amino-ethylphosphonic acid dimethylester;
N,N-dichloro-1-amino-1-methylethane phosphonic acid;
N,N-dichloro-1-amino-2-methylethane phosphonic acid;
N,N-dichloro-1-amino-2-methylpropane phosphonic acid;
N,N-dichloro-leucine phosphonic acid;
N,N-dichloro-4-amino-4-phosphonobutyric acid;
(±) N,N-dichloro-2-amino-5-phosphonovaleric acid;
N,N-dichloro-(+)-2-amino-5-phosphonovaleric acid;
N,N-dichloro d,1-2-amino-3-phosphonopropionic acid;
N,N-dichloro-2-amino-8-phosphonooctanoic acid;
N,N-dichloro-leucine boronic acid;
N,N-dichloro-β-alanine boronic acid;
N-chlorotaurine;
N-chloro-2-methyltaurine;
N-chloro-2,2,3,3-tetramethyl-β-alanine;
N-chloro-2,2-dimethyltaurine;
N-chloro-1,1,2,2-tetramethyltaurine;
N-bromo-2,2-dimethyltaurine;
N-bromo-1,1,2,2-tetramethyltaurine;
N-iodotaurine;
N-chloro-3,3-dimethylhomotaurine;
N-chloro-2-methyl-2-amino-ethanesulfonic acid; and
N-chloro-1-methyl-ethanesulfonic acid,
N-chloro amino-trimethylene phosphonic acid;
N-bromo-2-amino-5-phosphonopantanoic acid;
N-chloro amino-ethylphosphonic acid diesters, such as the diethylester;
N-chloro-1-amino-1-methylethane phosphonic acid;
N-chloro-1-amino-2-methylethane phosphonic acid;
N-chloro-1-amino-2-methylpropane phosphonic acid;
N-chloro-leucine phosphonic acid;
N-chloro-4-amino-4-phosphonobutyric acid;
(±) N-chloro-2-amino-5-phosphonovaleric acid;
N-chloro-(+)-2-amino-5-phosphonovaleric acid;
N-chloro d,1-2-amino-3-phosphonopropionic acid;
N-chloro-2-amino-8-phosphonooctanoic acid;
N-chloro-leucine boronic acid;
N-chloro-β-alanine boronic acid;
(1-(dichloroamino)cyclohexyl)methanesulfonic acid;
(1-(chloroamino)cyclohexyl)methanesulfonic acid;
2-(chloroamino)-N,N,N-2-tetramethylpropan-1-ammonium chloride;
2-(dichloroamino)-N,N,N-2-tetramethylpropan-1-ammonium chloride;
3-(chloroamino)-N,N,N-3-tetramethylbutan-1-ammonium chloride;
3-(dichloroamino)-N,N,N-3-tetramethylbutan-1-ammonium chloride;
3-(chloroamino)-N,N,N-triethyl-3-methylbutan-1-aminium chloride;
3-(dichloroamino)-N,N,N-triethyl-3-methylbutan-1-aminium chloride;
1-(2-(dichloroamino)-2-methylpropyl)-1-methylpiperidinium chloride;
1-(2-(chloroamino)-2-methylpropyl)-1-methylpiperidinium chloride;
(2-(dichloroamino)-2-methylpropyl)dimethylsulfonium chloride;
(2-(chloroamino)-2-methylpropyl)dimethylsulfonium chloride;
(4-(dichloroamino)-4-methylpentyl)trimethylphosphonium chloride;
(4-(chloroamino)-4-methylpentyl)trimethylphosphonium chloride;
3-(3-(dichloroamino)-3-methylbutylsulfonyl)-N,N,N-trimethylpropan-1-aminium chloride;
3-(3-(chloroamino)-3-methylbutylsulfonyl)-N,N,N-trimethylpropan-1-aminium chloride;
2-(3-(dichloroamino)-3-methylbutylsulfonyl)-N,N,N-trimethylethanaminium chloride;
2-(3-(chloroamino)-3-methylbutylsulfonyl)-N,N,N-trimethylethanaminium chloride;
1-(3-(chloroamino)-3-methylbutyl)-4,4-difluoro-1-methylpiperidinium chloride;
1-(3-(dichloroamino)-3-methylbutyl)-4,4-difluoro-1-methylpiperidinium chloride;
1-(3-chloro-4-methyl-2-oxooxazolidin-4-yl)-N,N,N-trimethylmethanaminium chloride;
(3-chloro-4-methyl-2-oxooxazolidin-4-yl)methanesulfonic acid;
(3-chloro-5-methyl-2-oxooxazolidin-5-yl)methanesulfonic acid;
4-(3-(chloroamino)-3-methylbutylsulfonyl)butanoic acid;
4-(3-(dichloroamino)-3-methylbutylsulfonyl)butanoic acid;
3-(3-(chloroamino)-3-methylbutylsulfonyl)propylphosphonic acid;
3-(3-(dichloroamino)-3-methylbutylsulfonyl)propylphosphonic acid;
(3-chloro-4,4-dimethyl-2-oxooxazolidin-5-yl)methanesulfonic acid;
2-(3-chloro-4,4-dimethyl-2,5-dioxoimidazolidin-1-yl)ethanesulfonic acid;
1-chloro-2,2,5,5-tetramethylimidazolidin-4-one;
1,3-dichloro-2,2,5,5-tetramethylimidazolidin-4-one;
1-bromo-3-chloro-2,2,5,5-tetramethylimidazolidin-4-one;
1,3-dibromo-2,2,5,5-tetramethylimidazolidin-4-one;
1,3-dichloro-2,5-bis(pentamethylene)imidazolidin-4-one;
1,3-dichloro-2-pentamethylene-5,5-dimethylimidazolidin-4-one;
1,3-dichloro-2,2-dimethyl-5-pentamethyleneimidazolidin-4-one;
1,3-dichloro-2,2,5-trimethyl-5-ethylimidazolidin-4-one;
1,3-dichloro-2-hydroxy-2,5,5-trimethylimidazolidin-4-one;
3-chloro-4,4-dimethyl-2-oxazolidinone;
3-chloro-4-ethyl-4-methyl-2-oxazolidinone; and
3-chloro-5,5-dimethyl-2-oxazolidinone.

10. The method of claim 1 wherein the infection is a fungal infection.

11. A method of claim 1 wherein the pharmaceutical composition further comprises a water-swellable polymer.

12. The method of claim 11 wherein the water-swellable polymer is a poly(ethylene oxide).

13. The method of claim 11 wherein the water-swellable polymer is a polyacrylic acid.

14. The method of claim 1 wherein the pharmaceutical composition further comprises a carrier, excipient, film-forming agent, humectant, penetration enhancer, plasticizer, solvent, co-solvent, plasticizer, and surfactant.

15. The method of claim 1 wherein the pharmaceutical composition is in the form of a lacquer.

16. The method of claim 1, further comprising administering to the subject an agent selected from the group consisting of antifungal, antibacterial, and antiviral agents.

17. The method of claim 1 wherein the compound is at least 90% stable for at least 30 days at about 25° C.

18. The method of claim 1 wherein the compound penetrates a nail within about 120 hours.

19. The method of claim 1 wherein the compound has at least two of the following properties:
(a) a stability of at least 90% for at least 30 days at 25° C.;
(b) a molecular weight between about 100 and about 300 Da;
(c) a water solubility between about 0.1 mg/ml and about 1 g/ml in octanol-saturated water;
(d) a log P value between about −1 and about 3;
(e) penetration of a nail within about 120 hours.

20. An apparatus for the treatment of a nail infection comprising an active agent comprising an N-halogenated or N,N-dihalogenated amine compound.

21. The apparatus of claim 20, further comprising a patch, wherein the patch comprises:
a backing medium; and
an adhesive matrix layer deposited thereon, the adhesive matrix layer comprising the active agent dissolved or dispersed in the adhesive matrix layer; and
optionally at least one penetration enhancer.
